(19) United States
(12) Patent Application Publication
(10) Pub. No.: US 2015/0164824 A1
(43) Pub. Date: Jun. 18, 2015

(54) METHODS AND COMPOSITIONS USEFUL FOR IMPROVING BONE AND JOINT HEALTH

(71) Applicant: ABBOTT LABORATORIES, Abbott Park, IL (US)

(72) Inventors: Tapas Das, Worthington, OH (US); Bindya Jacob, Singapore (SG)

(73) Assignee: ABBOTT LABORATORIES, Abbott Park, IL (US)

(21) Appl. No.: 14/421,564

(22) PCT Filed: Aug. 14, 2013
(86) PCT No.: PCT/US2013/054947

§ 371 Date: Feb. 13, 2015

Related U.S. Application Data
(60) Provisional application No. 61/682,906, filed on Aug. 14, 2012.

(51) Int. Cl. A61K 31/122; A61K 33/06; A61K 31/593; A23L 1/29; A61K 31/12

(52) U.S. Cl. CPC A61K 31/122; A23L 1/296; A61K 31/12; A61K 31/593; A61K 33/06; A23V 2002/00

(57) ABSTRACT

Compositions for improving bone health, joint health, or both in an individual in need thereof are provided, along with methods of using the compositions. Generally, the compositions include a combination of Curcumin and Vitamin K2. The compositions may also include a protein, a carbohydrate, and a fat. The compositions and methods may be particularly useful in the treatment or prevention of osteoarthritis, rheumatoid arthritis, systemic lupus erythematosus, osteopenia, and osteoporosis.

### CROSS REFERENCE TO RELATED APPLICATIONS

This application claims priority to and the benefit of U.S. Provisional Patent Application No. 61/682,906, filed Aug. 14, 2012, the entire content of which is incorporated by reference herein.

### FIELD

The general inventive concepts relate to methods and compositions for improving bone health, joint health, or both in an individual in need thereof. More particularly, the general inventive concepts relate to methods and compositions including a combination of Curcumin and Vitamin K2 for improving bone health, joint health, or both in an individual in need thereof.

### BACKGROUND

Millions of individuals suffer damage to joint and bone tissues, either from the normal bumps and bruises of everyday life, or as a result of various diseases and related conditions. Osteoarthritis, rheumatoid arthritis, osteopenia, and osteoporosis generally represent the most prevalent diseases influencing both bone health and joint health, although other diseases may also present problems in this area. Furthermore, other diseases not generally associated with bone health or joint health, such as systemic lupus erythematosus (SLE), may affect bones or joint structure and function. As the population continues to age, the number of individuals having bone and joint issues will undoubtedly continue to increase.

Recently, the Surgeon General reported that over 1.5 million osteoporotic fractures occur in the United States each year, leading to more than 500,000 hospitalizations, over 800,000 emergency room encounters, more than 2.6 million physician office visits, and the placement of nearly 180,000 individuals into nursing homes. These fractures are not only self-limiting and in some instances, debilitating, to the individual, but caring for these fractures is expensive and may require long term care.

Moreover, many conditions of impaired bone health or joint health are, or become, chronic in nature, thereby necessitating long term therapies. As such, there is a continuing need for a long term, safe means of effectively improving bone health, joint health, or both in individuals.

### SUMMARY

The general inventive concepts relate to methods and compositions for improving bone health, joint health, or both in an individual in need thereof. By way of example to illustrate various aspects of the general inventive concepts, several exemplary embodiments of methods and compositions (e.g., nutritional compositions) are provided herein.

In one exemplary embodiment, a composition comprising Curcumin and Vitamin K2 for use in treating or preventing osteoarthritis, rheumatoid arthritis, systemic lupus erythematosus (SLE), osteopenia, or osteoporosis in an individual in need thereof is provided. Use of the composition comprising Curcumin and Vitamin K2 may inhibit one or more of osteoclast differentiation, collagen degradation, and bone resorption. In one exemplary embodiment, the composition is a nutritional composition.

In one exemplary embodiment, the composition comprising Curcumin and Vitamin K2 for use in treating or preventing osteoarthritis, rheumatoid arthritis, SLE, osteopenia, or osteoporosis in an individual in need thereof comprises about 0.001% to about 3.4% of Curcumin by weight of the composition and about 0.0001% to about 0.1% of Vitamin K2 by weight of the composition.

In exemplary embodiments as described herein, an effective amount of each of the components of the composition may be provided. For example, the composition may comprise about 0.001% to about 3.4% of Curcumin by weight of the composition and about 0.0001% to about 0.1% of Vitamin K2 by weight of the composition. In one exemplary embodiment, the Curcumin is provided as bioavailable Curcumin and the Vitamin K2 is menaquinone-7.

In one exemplary embodiment, the composition further comprises Vitamin D3 and calcium. In one exemplary embodiment, the composition comprises, per serving or dose, about 160 IU to about 1,000 IU of Vitamin D3 and about 150 mg to about 800 mg of calcium.

In one exemplary embodiment, the composition further comprises one or more of at least one source of protein, at least one source of carbohydrate, and at least one source of fat. In one exemplary embodiment, the composition comprises about 1% to about 30% of at least one source of protein by weight of the composition, about 10% to about 80% of at least one source of carbohydrate by weight of the composition, and about 0.5% to about 30% of at least one source of fat by weight of the composition.

In one exemplary embodiment, the composition is a liquid nutritional product. In one exemplary embodiment, the composition is a reconstitutable powder. In one exemplary embodiment, the composition is a solid nutritional product.

In one exemplary embodiment, a nutritional composition for improving bone health, joint health, or both in an individual in need thereof is provided. The nutritional composition comprises: about 1% to about 30% of at least one source of protein by weight of the composition; about 0.001% to about 3.4% of Curcumin by weight of the composition; and about 0.0001% to about 0.1% of Vitamin K2 by weight of the composition.

In one exemplary embodiment, the nutritional composition further comprises, per serving or dose, about 160 IU to about 1,000 IU of Vitamin D3 and about 150 mg to about 1,000 mg of calcium.

In one exemplary embodiment, a composition comprising Curcumin and Vitamin K2 for use in improving bone health, joint health, or both in an individual in need thereof is provided. Use of the composition comprising Curcumin and Vitamin K2 may inhibit one or more of osteoclast differentiation, collagen degradation, and bone resorption to thereby improve bone health, joint health, or both in the individual. In one exemplary embodiment, the composition is a nutritional composition.

In one exemplary embodiment, a method for maintaining bone quality in an individual in need thereof is provided. The method includes administering to the individual in need thereof a nutritional composition comprising at least one source of protein in an amount sufficient to provide 5 grams to 50 grams of protein per serving, Curcumin, Vitamin K2, Vitamin D3, and calcium. Upon consumption of the nutritional composition, the bone quality of the individual in need thereof is maintained.

In one exemplary embodiment, a method for reducing bone loss in an individual in need thereof is provided. The method includes administering to the individual in need thereof a nutritional composition comprising at least one source of protein in an amount sufficient to provide 5 grams to 50 grams of protein per serving, Curcumin, Vitamin K2, Vitamin D3, and calcium. Upon consumption of the nutritional composition, the bone loss of the individual in need thereof is reduced.

In one exemplary embodiment, a method of inhibiting osteoclast differentiation in an individual in need thereof is provided. The method comprises administering to the individual in need thereof a composition comprising Curcumin and Vitamin K2. In one exemplary embodiment, the composition is a nutritional composition.

In one exemplary embodiment, a method of inhibiting collagen degradation in an individual in need thereof is provided. The method comprises administering to the individual in need thereof a composition comprising Curcumin and Vitamin K2. In one exemplary embodiment, the composition is a nutritional composition.

In one exemplary embodiment, a method of inhibiting bone resorption in an individual in need thereof is provided. The method comprises administering to the individual in need thereof a composition comprising Curcumin and Vitamin K2. In one exemplary embodiment, the composition is a nutritional composition.

### BRIEF DESCRIPTION OF THE FIGURES

FIG. 1 depicts a bar graph representing the relative differentiation of osteoclast precursors as evaluated in Example 1.

FIG. 2 depicts a bar graph representing the relative percentage inhibition of bone resorption by osteoclasts as evaluated in Example 2.

FIG. 3 depicts the maximal load (in Newtons) measured by 3-point bending of rat dam femurs 8 weeks after ovariectomization (Ovx) and nutritional intervention as evaluated in Example 11.

FIG. 4 depicts the ultimate load to failure (in Newtons) measured by 3-point bending of rat dam femurs 8 weeks after ovariectomization (Ovx) and nutritional intervention as evaluated in Example 11.

FIG. 5 depicts a bar graph representing the bone mineral density (BMD) measured by dual energy X-Ray absorptiometry (DXA) of rat dam lumbar vertebrae (L4-L5) before ovariectomization (Ovx) and 8 weeks after Ovx and nutritional intervention as evaluated in Example 11.

FIG. 6 depicts a bar graph representing the bone mineral density (BMD) measured by micro-CT (ex vivo) of rat dam femurs and lumbar vertebra (L5) 8 weeks after ovariectomization (Ovx) and nutritional intervention as evaluated in Example 11.

FIG. 7 depicts a bar graph representing the scores of certain components of the Modified Mankin's scoring system for assessing joint degradation of rat dam knee joints 8 weeks after ovariectomization (Ovx) and anterior cruciate ligament tear (ACLT) surgery and 9 weeks of nutritional intervention as evaluated in Example 12.

FIG. 8 depicts a bar graph representing the total score of the Modified Mankin's scoring system for assessing joint degradation of rat dam knee joints 8 weeks after ovariectomization (Ovx) and anterior cruciate ligament tear (ACLT) surgery and 9 weeks of nutritional intervention as evaluated in Example 12.

FIG. 9 depicts histology images of knee joints 8 weeks after ovariectomization (Ovx) and anterior cruciate ligament tear (ACLT) surgery and after 9 weeks of nutritional intervention as evaluated in Example 12, with the images shown in FIGS. 9A and 9B being representative of rat dams administered vehicle, calcium, and Vitamin D3, and the images shown in FIGS. 9C and 9D being representative of rat dams administered Curcumin, Vitamin K2, calcium, and Vitamin D3.

### DETAILED DESCRIPTION OF THE DISCLOSURE

While the general inventive concepts are susceptible of embodiment in many different forms, described herein in detail are specific embodiments thereof with the understanding that the present disclosure is to be considered as an exemplification of the principles of the general inventive concepts. Accordingly, the general inventive concepts are not intended to be limited to the specific embodiments illustrated and described herein.

The general inventive concepts described herein generally relate to methods and compositions for improving bone health, joint health, or both in an individual through the supplementation of the individual's diet with a combination of Curcumin and Vitamin K2. The exemplary methods and compositions described herein may be useful in treating or preventing diseases and conditions that affect bone health, joint health, or both, such as, for example, osteoarthritis, rheumatoid arthritis, osteopenia, and osteoporosis. The exemplary methods and compositions described herein may be useful for inhibiting one or more of osteoclast differentiation, collagen degradation, and bone resorption to thereby provide effective treatment for diseases and conditions that affect bone health, joint health, or both. The exemplary methods and compositions described herein may be useful in maintaining bone quality, reducing bone loss, or both in an individual in need thereof. Accordingly, the exemplary methods and compositions described herein may be useful in improving the overall bone health, joint health, or both in an individual.

The terminology as set forth herein is for description of the exemplary embodiments only and should not be construed as limiting the disclosure as a whole. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise. Additionally, recitations of numerical ranges by endpoints include all numbers subsumed within that range (e.g., 1 to 5 includes 1, 1.5, 2, 2.75, 3, 3.80, 4, 5). As used herein, the term “about,” when preceding a numerical value or range includes the exact numerical value or range recited, as well as values within 10% above or below the recited numerical value or range.

The terms “nutritional product” and “nutritional composition,” as used herein, are used interchangeably and, unless otherwise specified, refer to nutritional liquids, nutritional powders, nutritional bars, nutritional supplements, and any other nutritional product as known in the art. The nutritional powders may be reconstituted to form a nutritional liquid. The nutritional product or nutritional composition may include one or more of at least one source of protein, at least one source of carbohydrate, and at least one source of fat, and is suitable for oral consumption by a human.

The terms “nutritional liquid” and “liquid nutritional product,” as used herein, unless otherwise specified, refer to a nutritional composition in ready-to-drink liquid form, concentrated form, and a nutritional liquid made by reconstituting a nutritional powder prior to use.

The terms “nutritional powder” an “reconstitutable powder,” as used herein, unless otherwise specified, refer to a nutritional composition in flowable or scoopable form that can be reconstituted with water or another aqueous liquid prior to consumption and include both spray dried and dry mixed/dry blended powders.

The term “nutritional semi-solid,” as used herein, unless otherwise specified, refers to a nutritional composition that is intermediate in properties, such as rigidity, between solid and liquid. Some semi-solid examples include, but are not limited to, puddings, yogurts, gels, gelatins, and doughs.

The term “nutritional semi-liquid,” as used herein, unless otherwise specified, refers to a nutritional composition that is intermediate in properties, such as flow properties, between liquid and solid. Some semi-liquid examples include, but are not limited to, thick shakes, liquid yogurts, and liquid gels.

The term “Curcumin,” as used herein, unless otherwise specified, refers to Curcumin, bioavailable Curcumin, and derivatives and analogs thereof.

The term “bioavailable,” as used herein, unless otherwise specified, refers to the ability of a compound to enter into and remain in the bloodstream of an individual such that the substance can be absorbed into cells in the body. As the degree of bioavailability of a compound increases, the compound becomes more likely to enter into and remain in the bloodstream where it can be absorbed and used by the body. As the degree of bioavailability of a compound decreases, the compound becomes more likely to go directly into the gastrointestinal area and be expelled from the body before entering the bloodstream.

The term “effective amount,” as used herein, unless otherwise specified, refers to a sufficient amount of a composition or an agent (e.g., Curcumin, Vitamin K2) to facilitate a desired therapeutic effect (e.g., maintain or improve bone health, joint health, or both) in an individual. The exact amount required will vary from individual to individual, for example, based on the species, age, weight, lifestyle and general condition of the particular individual.

The term “bone quality,” as used herein, unless otherwise specified, refers to characteristics that provide an indication of bone health. For example, bone quality includes characteristics such as bone mineral density (BMD), bone strength, bone mineral content (BMC), bone microarchitecture, accumulated microscopic damage, bone turnover, and so forth.

The term “administering,” as used herein, unless otherwise specified, should be understood to include providing a composition to an individual, the act of consuming the composition by the individual, and combinations thereof.

The term “serving,” as used herein, unless otherwise specified, is intended to be construed as any amount which is intended to be consumed by an individual in one sitting or within one hour or less. In one exemplary embodiment, a serving of a nutritional powder is about 40 grams of nutritional powder, which may be reconstituted with, for example, 8 fl oz (1 cup) of a suitable liquid (e.g., water, milk).

The term “individual,” as used herein, unless otherwise specified, refers to a mammal including companion animals, livestock, laboratory animals, working animals, sport animals, and humans. In certain exemplary embodiments, the individual is a human.

The term “elderly,” as used herein, unless otherwise specified, refers to a human of at least 45 years of age, including at least 50 years of age, at least 55 years of age, at least 60 years of age, at least 65 years of age, at least 70 years of age, at least 75 years of age, and including at least 80 years of age or greater. The term “elderly” also includes humans of 45 years of age to 100 years of age, and humans of 55 years of age to 80 years of age.

The term “individual in need thereof,” as used herein, unless otherwise specified, refers to an individual diagnosed as having, or exhibiting symptoms associated with, one or more of osteoporosis, osteoarthritis, osteopenia, and rheumatoid arthritis. In certain exemplary embodiments, the individual in need thereof has degenerated cartilage in one or both knees. In certain exemplary embodiments, the individual in need thereof is diagnosed as having, or exhibiting symptoms associated with, low-grade inflammation. In certain exemplary embodiments, the individual in need thereof is a menopausal or postmenopausal woman. In certain exemplary embodiments, the individual in need thereof is estrogen deficient. In certain exemplary embodiments, the individual in need thereof is an elderly human.

The terms “susceptible” and “at risk,” as used herein, unless otherwise specified, mean having little resistance to a certain condition or disease (e.g., osteoporosis, osteoarthritis, rheumatoid arthritis), including one or more of being genetically predisposed to the condition or disease, having a family history of the condition or disease, and having symptoms of the condition or disease or a precursor thereof.

The term “shelf stable,” as used herein, unless otherwise specified, refers to a nutritional liquid that remains commercially stable after being packaged and contained within a hermetically sealed container and then stored at 18-24° C. for at least 3 months, including about 6 months to about 24 months, and also including about 12 months to about 18 months.

All percentages, parts, and ratios, as used herein, are by weight of the total composition, unless otherwise specified. All such weights as they pertain to listed ingredients are based on the active level and, therefore, do not include solvents or by-products that may be included in commercially available materials, unless otherwise specified.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The various exemplary compositions described herein may also be substantially free of any optional or selected essential ingredient or feature described herein, provided that the composition still contains all of the required ingredients or features as described herein. In this context, and unless otherwise specified, the term “substantially free” means that the selected composition contains less than a functional amount of the optional or selected ingredient, typically less than 0.5%, including less than 0.25%, including less than 0.1%, and also including zero percent, by weight, of such optional or selected ingredient.

The exemplary methods may use and the exemplary compositions may comprise, consist of, or consist essentially of the elements of the compositions as described herein, as well as any additional or optional element described herein or otherwise known (now or in the future) to be useful in certain exemplary applications.

Methods and nutritional compositions for improving bone health, joint health, or both are provided herein. The exemplary methods described herein include administering a composition comprising Curcumin and Vitamin K2 to an individual in need thereof. In one exemplary embodiment, the method includes administering a composition comprising an effective amount of Curcumin and an effective amount of Vitamin K2 to an individual in need thereof. In one exemplary embodiment, the compositions comprise an effective amount of Curcumin and an effective amount of Vitamin K2. As will be explained in more detail below, the inventors discovered that fortifying an individual's diet with a combination of Curcumin and Vitamin K2 can result in improved bone health, bone strength, and joint health. Particularly, the combination of Curcumin and Vitamin K2 has been found to synergistically inhibit osteoclast differentiation, and consequently collagen degradation, such that overall bone health is improved. Further, the combination of Curcumin and Vitamin K2 has been found to be effective in suppressing bone resorption, increasing bone mineral density, and increasing bone strength. Accordingly, the combination of Curcumin and Vitamin K2 may be effective for treating, reducing symptoms of, or controlling diseases and conditions associated with or that otherwise affect bone health, joint health, or both including, but not limited to, osteoarthritis, rheumatoid arthritis, SLE, osteopenia, and osteoporosis.

The exemplary methods and compositions described herein offer individuals in need of improved bone health, joint health, or both a simple and effective means for improving overall bone health and joint health. The features of the exemplary methods and compositions, as well as some of the many optional variations and additions, are described in more detail hereafter.

### Product Form

The exemplary compositions described herein may be formulated and administered in any known or otherwise suitable oral product form. Any solid, semi-solid, liquid, semi-liquid, or powder form, including combinations or variations thereof, are suitable for use herein, provided that such forms allow for safe and effective oral delivery to the individual of the exemplary compositions as described herein. The exemplary compositions may be formulated to include only the essential ingredients described herein, or may be modified with optional ingredients to form a number of different product formulations.

As previously discussed, the exemplary compositions described herein comprise Curcumin and Vitamin K2. In one exemplary embodiment, the composition comprises an effective amount of Curcumin and an effective amount of Vitamin K2. In one exemplary embodiment, the composition comprising Curcumin and Vitamin K2 comprises about 0.001% to about 3.4% of Curcumin by weight of the composition and about 0.0001% to about 0.1% of Vitamin K2 by weight of the composition.

In one exemplary embodiment, the composition comprising a combination of Curcumin and Vitamin K2 is a nutritional composition. In one exemplary embodiment, the nutritional composition comprising a combination of Curcumin and Vitamin K2 further comprises one or more of at least one source of protein, at least one source of carbohydrate, and at least one source of fat. In one exemplary embodiment, the nutritional composition comprising a combination of Curcumin and Vitamin K2 and one or more of at least one source of protein, at least one source of carbohydrate, and at least one source of fat may further comprise vitamins, minerals, or combinations thereof.

In one exemplary embodiment, the nutritional composition comprises about 1% to about 30% of at least one source of protein by weight of the composition, about 0.001% to about 3.4% of Curcumin by weight of the composition, and about 0.0001% to about 0.1% of Vitamin K2 by weight of the composition. In one exemplary embodiment, the nutritional composition further comprises, per serving or dose, about 160 IU to about 1,000 IU of Vitamin D3 and about 150 milligrams to about 1,000 milligrams of calcium. In one exemplary embodiment, the Curcumin is provided as bioavailable Curcumin and the Vitamin K2 is menaquinone-7.

The exemplary nutritional compositions may be formulated with sufficient kinds and amounts of nutrients to provide a sole, primary, or supplemental source of nutrition, or to provide a specialized nutritional composition, including specialized nutrition to individuals in need of bone health improvement, joint health improvement, or both. In certain exemplary embodiments, the nutritional composition provides up to 500 kcal of energy per serving, including about 20 kcal to about 500 kcal, about 75 kcal to about 500 kcal, about 150 kcal to about 500 kcal, about 200 kcal to about 500 kcal, about 300 kcal to about 500 kcal, about 350 kcal to about 500 kcal, or about 400 kcal to about 500 kcal per serving.

### Nutritional Solids

In one exemplary embodiment, the composition comprising a combination of Curcumin and Vitamin K2 is formulated as a solid nutritional product. Exemplary forms of the solid nutritional product include, but are not limited to, tablets; pellets; extruded solids; snack and meal replacement products, including those formulated as bars, sticks, or baked goods such as cookies, breads, or cakes; frozen liquids; candy; breakfast cereals; powders, granulated solids, or other particulates; snack chips or bites; frozen or retorted entrees; and so forth. In one exemplary embodiment, when the composition comprising a combination of Curcumin and Vitamin K2 is formulated as a solid nutritional product, the serving is within a range of 25 grams to 150 grams. In one exemplary embodiment, when the composition comprising a combination of Curcumin and Vitamin K2 is formulated as a solid nutritional product, the individual is administered one to four servings per day of the solid nutritional product.

### Nutritional Powders

In one exemplary embodiment, the composition comprising a combination of Curcumin and Vitamin K2 is formulated as a reconstitutable powder. Exemplary reconstitutable powders may be spray dried, agglomerated, or dry blended powder compositions. Such exemplary reconstitutable powders generally can be easily scooped and measured with a spoon or similar device, wherein the powder can be easily reconstituted by the intended user with a suitable aqueous liquid, such as water or milk, to form a nutritional composition for immediate oral or enteral use. In this context, “immediate use” generally means within about 48 hours, most typically within about 24 hours, preferably right after reconstitution. The quantity of a reconstitutable powder required to produce a volume suitable for one serving can vary. In certain exemplary embodiments, a serving of the reconstitutable powder ranges from about 25 grams to about 50 grams, including about 30 grams to about 45 grams, and also including about 35 grams to about 40 grams.

### Nutritional Liquids

In one exemplary embodiment, the composition comprising a combination of Curcumin and Vitamin K2 is formulated as a liquid nutritional product. Exemplary forms of the liquid nutritional product include, but are not limited to, concentrated and ready-to-feed nutritional liquids, snack, and meal replacement products; hot or cold beverages; carbonated or non-carbonated beverages; juices or other acidified beverages; milk or soy-based beverages; shakes; coffees; teas; and so forth. Typically, the liquid nutritional product is formulated as a suspension, an emulsion, or a clear or substantially clear liquid.

Exemplary nutritional emulsions suitable for use may be aqueous emulsions comprising protein, carbohydrate, and fat. These emulsions are generally flowable or drinkable liquids at about 1° C. to about 25° C. and are typically in the form of oil-in-water, water-in-oil, or complex aqueous emulsions, although such emulsions are typically in the form of oil-in-water emulsions having a continuous aqueous phase and a discontinuous oil phase.

The exemplary nutritional emulsions may be, and typically are, shelf stable. The exemplary nutritional emulsions typically contain up to 95% by weight of water, including about 50% to about 95%, also including about 60% to about 90%, and also including about 70% to about 85%, of water by weight of the nutritional emulsions. The exemplary nutritional emulsions may have a variety of product densities, but typically have a density greater than 1.03 g/ml, including greater than 1.04 g/ml, and including greater than 1.055 g/ml; or a density between about 1.06 g/ml and about 1.12 g/ml, and also including between about 1.085 g/ml and about 1.10 g/ml.

In one exemplary embodiment, the nutritional emulsion may have a pH between about 3.5 and about 8, and more advantageously between about 4.5 and about 7.5, including about 5.5 to about 7.3, and including about 6.2 to about 7.

In one exemplary embodiment, the composition is a nutritional liquid formulated as a clear liquid having a pH of about 2 to about 6, and also having no more than 0.5% fat by weight of the composition. The limited amount of fat contributes to the desired clarity and pH of the clear nutritional liquid. Typically, liquid nutritional compositions desired to be clear, or at least considerably translucent, are substantially free of fat. As used herein “substantially free of fat” refers to a nutritional composition containing less than 0.5%, including less than 0.1%, fat by weight of the composition. “Substantially free of fat” also may refer to an exemplary nutritional composition disclosed herein that contains no fat, i.e., zero fat. Furthermore, exemplary embodiments of nutritional liquids that have an acidic pH in the range of about 2 to about 6 (e.g., juices, fruit juices, fruit-flavored beverages) are typically substantially free of fat. Typically, nutritional liquids that are both clear and have a pH between about 2 to about 6 are also typically substantially free of fat. In certain exemplary embodiments, the pH of the nutritional liquid may be between 2.5 and 4.6, including between 3 and 3.5. In certain exemplary embodiments, where the nutritional liquid is substantially free of fat but has some amount of fat present, the fat may be present as a result of being inherently present in another ingredient (e.g., a source of protein) or may be present as a result of being added as one or more separate sources of fat. Additionally, in certain exemplary embodiments where the composition is a nutritional liquid having a pH of about 2 to about 6, certain protein sources are more or less suitable for use in formulating the nutritional liquid. For example, whey protein isolate, whey protein concentrate, whey protein hydrolysate, hydrolyzed casein, hydrolyzed soy protein, hydrolyzed pea protein, and commercially available soluble soy protein isolates are generally more suitable for use in nutritional liquids having a pH of about 2 to about 6.

In certain exemplary embodiments, when the composition comprising a combination of Curcumin and Vitamin K2 is formulated as a liquid nutritional product, a serving thereof may be within a range of about 30 milliliters to about 500 milliliters (˜1 fl oz to ˜17 fl oz). In certain exemplary embodiments, when the composition comprising a combination of Curcumin and Vitamin K2 is formulated as a liquid nutritional product, a serving thereof may be about 237 milliliters (˜8 fl oz). In certain other exemplary embodiments, when the composition comprising a combination of Curcumin and Vitamin K2 is formulated as a liquid nutritional product, a serving thereof may be within a range of about 150 milliliters to about 500 milliliters (˜5 fl oz to ˜17 fl oz), within a range of about 177 milliliters to about 417 milliliters (˜6 fl oz to ˜14 fl oz), or within a range of about 207 milliliters to about 296 milliliters (˜7 fl oz to ˜10 fl oz). In still other exemplary embodiments, when the composition comprising a combination of Curcumin and Vitamin K2 is formulated as a liquid nutritional product, a serving thereof may be within a range of about 30 milliliters to about 75 milliliters (˜1 fl oz to ˜2.5 fl oz). In certain exemplary embodiments, when the composition comprising a combination of Curcumin and Vitamin K2 is formulated as a liquid nutritional product, the individual is administered one to four servings per day of the liquid nutritional product.

### Curcumin

As used herein, the term “Curcumin” includes isolated Curcumin or analogues or derivatives thereof, as described herein, or any combinations thereof. Curcumin also refers to the compound having a systematic name of (1E,6E)-1,7-bis(4-hydroxy-3-methoxyphenyl)-1,6-heptadiene-3,5-dione and having the formula:

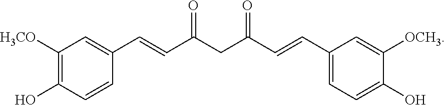 [SMILES: COc1cc(/C=C/C(=O)CC(=O)/C=C/c2ccc(O)c(OC)c2)ccc1O]

Curcumin is the principal Curcuminoid of turmeric. In addition to the keto form shown above, Curcumin may also be in enol form. Other naturally occurring Curcuminoids of turmeric include bis-demethoxyCurcumin (one methoxy group removed from Curcumin structure) and demethoxyCurcumin (both methoxy groups removed from Curcumin structure). Curcumin is a polyphenol and exhibits anti-inflammatory and antioxidant effects.

Conventionally, in some cases Curcumin has suffered lower bioavailability when taken orally, and thus when formulated at higher concentrations to counter its inherent poor bioavailability to achieve the desired systemic delivery, the products to which Curcumin is added may often take on an intense, undesirable yellow color. The term “Curcumin,” as used herein, includes Curcumin that has been formulated as “bioavailable Curcumin,” which may exhibit an improved bioavailability as compared to conventionally used Curcumin. As such, Curcumin formulated as bioavailable Curcumin can be utilized at lower concentrations in the exemplary methods and compositions described herein, while still maintaining its bone health and joint health promoting activity.

The term “bioavailable Curcumin” may refer to Curcumin and derivatives and analogs thereof, including natural and synthetic derivatives of Curcumin, as well as any combination of one or more of Curcumin, a Curcumin derivative, and a Curcumin analog that has been processed or otherwise manipulated to improve the bioavailability thereof. In particular, in one exemplary embodiment, the term “bioavailable Curcumin” may encompass compounds having a 1,7-bis(4-hydroxyphenyl)-1,6-heptadiene-3,5-dione skeleton, or a 1,7-bis(4-hydroxyphenyl) hept-4-en-3-one skeleton, wherein the phenyl groups independently may bear one or more alkoxy residues, especially one methoxy residue in the 3-position. In one exemplary embodiment, the composition comprising a combination of Curcumin and Vitamin K2 may further comprise additional Curcuminoids, such as demethoxyCurcumin and bisdemethoxyCurcumin. In one exemplary embodiment, when the composition further comprises demethoxyCurcumin, bisdemethoxyCurcumin, or both, the demethoxyCurcumin or bisdemethoxyCurcumin may be present as part of a complex with Curcumin.

The “bioavailable Curcumin” used in certain exemplary compositions herein shows improved oral bioavailability as compared to “non-bioavailable Curcumin.” The term “non-bioavailable Curcumin,” as used herein, refers to Curcumin that has not been processed or otherwise manipulated in an effort to improve the bioavailability thereof, and does not mean that the Curcumin has no bioavailability. The oral bioavailability can be determined in experiments involving oral administration of the bioavailable Curcumin (and administration of a corresponding amount of non-bioavailable Curcumin) to an individual and measuring the level of Curcumin in a biological sample obtained from the individual over time, wherein the biological sample may be derived from a body fluid, for example, serum, plasma, whole blood, or cerebrospinal fluid, or from a tissue (e.g., brain, liver, kidney, or heart). For analysis, the Curcumin level in the examined body fluid or tissue may be plotted against time, and the area under the curve (AUC), for example, the total area under the curve from t=0 (time of administration) to t=infinity (AUC0-infinity), or the area under the curve within a defined period (e.g. from t=0 to t=6 hours (AUC0-6H)) may be calculated. In general, a higher AUC relative to the AUC obtained by administration of non-bioavailable Curcumin indicates an improved bioavailability. The absolute bioavailability may be calculated from the resulting AUC data as a percentage based on the corresponding AUC data obtained from intravenous administration of Curcumin.

In one exemplary embodiment, the amount of Curcumin in the blood, determined as AUC0-6H after a single oral administration to a human or an animal subject, such as a rat, of a dose or serving of a composition containing bioavailable Curcumin corresponding to 20 milligrams of total Curcumin is significantly higher than after oral administration of the same amount of non-bioavailable Curcumin in the composition, including at least 2 times higher, at least 3 times higher, at least 4 times higher, at least 6 times higher, at least 8 times higher, at least 10 times higher, or at least 15 times higher, and, for example, up to 30 times higher.

As used herein, the amount of Curcumin in the blood being “significantly higher” means a statistically significant increase of this parameter in individuals after oral administration of 20 milligrams of bioavailable Curcumin in the exemplary compositions described herein as compared to the control 20 milligrams of non-bioavailable Curcumin. A statistical test known in the art, such as ANOVA or Student's t-test, may be used to determine the significance of this difference, wherein the p-value is at least <0.1, <0.5, <0.01, <0.005, <0.001 or <0.0001.

Curcumin can be prepared in a number of ways including, for example, using Meltrex® or similar melt-extrusion technology to prepare extruded solids and improve the bioavailability of the Curcumin as compared to Curcumin not produced by melt extrusion. Meltrex® or similar melt-extrusion technology methods are known in the art and can be applied to produce bioavailable Curcumin by one skilled in the art based on the disclosure herein. Accordingly, in one exemplary embodiment, the Curcumin used in the composition is melt-extruded Curcumin.

The term “melt-extruded Curcumin” as used herein refers to a melt-processed solid dispersion product comprising (a) one or more Curcuminoids, (b) a nutritionally acceptable thermoplastic polymer, and (c) a phosphatide.

The term “Curcuminoid,” as used herein, refers to Curcumin and derivatives thereof and analogs thereof, such as demethoxyCurcumin and bisdemethoxyCurcumin. These include natural and synthetic derivatives of Curcumin, and any combination of more than one Curcuminoid. In particular, for purposes herein, the term “Curcuminoid” may encompass compounds having a 1,7-bis(4-hydroxyphenyl)-1,6-heptadiene-3,5-dione skeleton, or a 1,7-bis(4-hydroxyphenyl) hept-4-en-3-one skeleton, wherein the phenyl groups independently may bear one or more alkoxy residues, especially one methoxy residue in the 3-position. An example of a suitable commercially available source of a mixture of Curcuminoids is “Curcumin C3 Complex®,” available from Sabinsa Corporation (East Windsor, N.J.).

The term “nutritionally acceptable,” as used herein, refers to a compound that does not cause acute toxicity when it is ingested or administered orally. All components of the melt-extruded Curcumin are nutritionally acceptable.

The nutritionally acceptable thermoplastic polymer used in the melt-extruded Curcumin described herein is a polymer capable of acting as a solid meltable solvent. It forms a matrix for dispersion, and in particular for dissolution, of the Curcuminoid. In some embodiments, the polymer is at least partly soluble or swellable in aqueous media, expediently under the conditions of use, and in particular under physiological conditions in the digestive tract. In an exemplary embodiment, the nutritionally acceptable thermoplastic polymer is a water-soluble polymer.

A wide range of nutritionally acceptable thermoplastic polymers may be utilized. For example, suitable nutritionally acceptable thermoplastic polymers include, but are not limited to, hydroxypropylmethylcellulose (Methocel™, Pharmacoat™), polymethacrylate (Eudragit™ EO), hydroxypropylcellulose (Klucel™), a polyvidone, or combinations thereof.

The term “phosphatide,” as used herein, refers to compounds which are derivatives of glycero-3 phosporic acid that contain at least one O-acyl, O-alkyl or O-alk-1′-enyl residue attached to the glycerol moiety and a polar head made of a nitrogenous base, a glycerol, or an inositol unit. The terms “phosphatide,” “glycerophospholipid,” and “phosphoglyceride” are used interchangeably. In an exemplary embodiment, the phosphatide utilized in the melt-extruded Curcumin is a lecithin. Lecithins are particular phosphatidylcholines, i.e., a group of phosphatides composed of phosphoric acid, choline, and fatty acids.

In certain exemplary embodiments, the melt-extruded Curcumin includes a normally solid polyol (i.e., “normally solid” means that the polyol is solid at NTP (Normal Temperature and Pressure, i.e., 20° C. and 1 atm)). The normally solid polyol acts as a melting point or softening point depressant and facilitates the uniform incorporation of the Curcuminoid into the solid dispersion matrix. The normally solid polyol may act as a plasticizer for the nutritionally acceptable polymer. In addition, the normally solid polyol may initially melt and the other component may dissolve in the melt.

In general, the melt-extruded Curcumin is utilized in certain exemplary compositions described herein comprises: a) about 0.1% to about 50%, for example, about 5% to about 30% or about 10% to about 20%, by weight of Curcuminoids; b) about 20% to about 95%, for example, about 40% to about 80%, by weight of nutritionally acceptable thermoplastic polymer; c) about 5% to about 50%, for example, about 5% to about 25%, by weight of phosphatide; and d) about 0% to about 50%, for example, about 1% to about 30% or about 5% to about 15%, by weight of normally solid polyol.

The melt-extruded Curcumin may be prepared by a method comprising: a) blending one or more Curcuminoids (e.g., Curcumin, demethoxyCurcumin, bisdemethoxyCurcumin), a nutritionally acceptable thermoplastic polymer, and a phosphatide; b) heating the blend to obtain a homogeneous melt; c) forcing the homogenous melt through one or more nozzles; and d) allowing the homogenous melt to solidify to obtain a melt-extruded curcumin product. Steps a) to c) may be performed in one or more than one suitable apparatus, such as an extruder or kneader extruder. In addition, in certain exemplary embodiments, the melt-extruded Curcumin may be milled or otherwise processed to provide the melt-extruded Curcumin in powder or granular form.

Further details regarding melt-extruded Curcumin and methods for preparing the same may be found in International Publication No. WO 2012/049253 A1, which is incorporated herein by reference in its entirety.

In one exemplary embodiment, Curcumin can be co-supplemented with piperine (generally extracted from black pepper) to increase the bioavailability and hence the absorbability of the Curcumin. In one exemplary embodiment, piperine is co-supplemented in an amount of about 20 milligrams to increase the bioavailability of the Curcumin. In one exemplary embodiment, the ratio of piperine to Curcumin is about 1:25 to about 1:5.

In one exemplary embodiment, Curcumin may be solubilized in an oil having an HLB (hydrophilic-lipophilic balance) of about 0.7 to about 14 (i.e., a polar oil) such that the resulting oil mixture provides increased bioavailability of the Curcumin. One suitable polar oil for dissolving the Curcumin is a medium chain triglyceride oil (MCT oil).

In one exemplary embodiment, the Curcumin is a mixture of Curcuminoids (e.g., Curcumin, demethoxyCurcumin, and bisdemethoxyCurcumin) obtained from the rhizomes of Curcuma Longa. In one exemplary embodiment, the Curcumin is melt-extruded Curcumin obtained using Meltrex® technology (Abbott Nutrition, Columbus, Ohio). In one exemplary embodiment, the Curcumin is Meriva Bioavailable Curcumin, commercially available from Idena SPA (Milan, Italy). In one exemplary embodiment, the Curcumin is Longvida Optimized Curcumin, commercially available from Verdure Sciences (Noblesville, Ind.). In one exemplary embodiment, the Curcumin is Theracurmin CR-011L, commercially available from Theravalues Corporation (Tokyo, Japan). In one exemplary embodiment, the Curcumin is Curqlife Curcumin, commercially available from Interhealth Nutraceuticals (Benicia, Calif.).

In one exemplary embodiment, the composition, including a nutritional composition based thereon based thereon, comprises about 0.001% to about 3.4% of Curcumin by weight of the composition. In certain exemplary embodiments, the composition comprises at least 0.001% of Curcumin by weight of the composition, including between about 0.002% and about 3.4%, including between about 0.002% and about 3.36%, including between about 0.005% and about 1.87%, including between about 0.03% and about 0.935%, including between about 0.1% and about 0.5%, including between about 0.1% to about 0.467%, and also including between about 0.234% and about 0.3%, by weight of the composition. Certain other exemplary embodiments of the composition comprise Curcumin in amounts between about 0.002% and about 0.234%, between about 0.005% and about 0.467%, between about 0.03% and about 0.935%, between about 0.1% and about 1.87%, and between about 0.3% and about 3.36%, by weight of the composition.

In one exemplary embodiment, the composition, including a nutritional composition based thereon, comprises Curcumin in an amount between about 1 milligram and about 10,000 milligrams per dose or serving of the composition. In one exemplary embodiment, the composition, including a nutritional composition based thereon, comprises Curcumin in an amount between about 50 milligrams and about 7,500 milligrams per dose or serving of the composition. In another exemplary embodiment, the composition, including a nutritional composition based thereon, comprises Curcumin in an amount between about 100 milligrams and about 5,000 milligrams, including between about 200 milligrams and about 4,000 milligrams, including between about 400 milligrams and about 2,000 milligrams, including between about 1,200 milligrams and about 1,800 milligrams per dose or serving of the composition.

As previously discussed, in one exemplary embodiment, the composition, including a nutritional composition based thereon based thereon, may include melt-extruded Curcumin. In certain exemplary embodiments, the composition includes melt-extruded Curcumin in an amount of about 20 milligrams to about 7,500 milligrams per serving, including about 150 milligrams to about 6,500 milligrams, including about 300 milligrams to about 6,000 milligrams, including about 400 milligrams to about 5,000 milligrams, including about 500 milligrams to about 2,500 milligrams, including about 750 milligrams to about 1,500 milligrams, and also including about 750 milligrams to about 1,000 milligrams per serving of the composition. As previously discussed, in certain exemplary embodiments, the amount of Curcuminoids contained in the melt-extruded Curcumin may be between about 0.1% and about 50% by weight of the melt-extruded Curcumin. In other exemplary embodiments the composition includes melt-extruded Curcumin in an amount sufficient to provide up to about 3,750 milligrams of Curcuminoids per serving, including between about 2 milligrams and about 3,750 milligrams, including between about 50 milligrams and about 3,000 milligrams, including between about 150 milligrams and about 2,000 milligrams, including between about 250 milligrams and about 1,500 milligrams, including between about 300 milligrams and about 1,000 milligrams, and also including between about 400 milligrams and about 750 milligrams of Curcuminoids per serving. Since the Curcuminoids in melt-extruded Curcumin are significantly more bioavailable than conventional crystalline Curcuminoids, a smaller amount of Curcuminoids are required to achieve the bone health and joint health effects described herein. For example, an effective amount of non-formulated crystalline Curcuminoids may be about 1,500 milligrams, whereas an effective amount of Curcumin formulated as bioavailable Curcumin may be about 150 milligrams (e.g., about 300 milligrams of melt-extruded Curcumin containing about 50% by weight Curcuminoids) due to a 10-fold increase in bioavailability.

In one exemplary embodiment, the composition, including a nutritional composition based thereon based thereon, comprises sufficient Curcumin to provide an individual with at least 1 milligram, including at least 3 milligrams, including between about 1 milligram and about 10,000 milligrams, including between about 10 milligrams and about 10,000 milligrams, including between about 100 milligrams and about 4,000 milligrams, including between about 400 milligrams and about 2,000 milligrams, including between about 1,200 milligrams and about 1,800 milligrams, per day of Curcumin. In certain exemplary embodiments, the total daily amount of Curcumin may be administered to an individual in a single undivided dose or serving of the composition, or may be split into multiple (e.g., two, three, four) doses or servings per day of the composition.

### Vitamin K2

As previously discussed, the exemplary compositions described herein also comprise Vitamin K2 in addition to Curcumin. Any suitable form of Vitamin K2, also referred to as menaquinone, may be utilized in the exemplary methods and compositions described herein. Vitamin K2 is a fat-soluble vitamin important for post-translational modification of certain proteins, mostly required for blood coagulation and metabolic pathways in bone.

Vitamin K2 is prevalent in organ meats, egg yolks, and dairy products. Further, a traditional Japanese food, natto, which consists of fermented soybeans produced by Bacillus subtilis natto, is uniquely rich in Vitamin K2, and particularly menaquinone-7 (MK-7). The formula for MK-7, also referred to as 2-methyl-3-all-trans-farnesyl digeranyl-1,4-naphthoquinone or by its systematic name (all-E)-2-(3,7,11,15,19,23,27-Heptamethyl-2,6,10,14,18,22,26-octacosaheptaenyl)-2-methyl-1,4-naphthalenedione, is shown below:

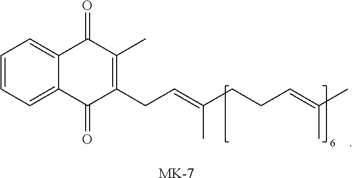

In certain exemplary embodiments, the composition, including a nutritional composition based thereon based thereon, comprises at least 0.0001% of Vitamin K2 by weight of the composition. In one exemplary embodiment, the composition, including a nutritional composition based thereon, comprises about 0.0001% to about 0.1% of Vitamin K2 by weight of the composition. In one exemplary embodiment, the composition, including a nutritional composition based thereon, comprises about 0.0005% to about 0.1% of Vitamin K2 by weight of the composition. In one exemplary embodiment, the composition, including a nutritional composition based thereon, comprises about 0.001% to about 0.1% of Vitamin K2 by weight of the composition. In one exemplary embodiment, the composition, including a nutritional composition based thereon, comprises about 0.0025% to about 0.1% by weight of the composition. In one exemplary embodiment, the composition, including a nutritional composition based thereon, comprises about 0.01% to about 0.1% of Vitamin K2 by weight of the composition. In one exemplary embodiment, the Vitamin K2 present in the composition, including a nutritional composition based thereon, is menaquinone-7 (MK-7). In one exemplary embodiment, the Vitamin K2 present in the composition, including a nutritional composition based thereon, is menaquinone-4 (MK-4). In one exemplary embodiment, the Vitamin K2 present in the composition, including a nutritional composition based thereon, is a combination of MK-7 and MK-4.

In one exemplary embodiment, the composition, including a nutritional composition based thereon, comprises Vitamin K2 (e.g., in the form of MK-7 or a combination of MK-7 and MK-4) in an amount between about 25 micrograms and 200 micrograms per dose or serving of the composition. In one exemplary embodiment, the composition, including a nutritional composition based thereon, comprises Vitamin K2 in an amount between about 50 micrograms and about 150 micrograms per dose or serving of the composition. In another exemplary embodiment, the composition, including a nutritional composition based thereon, comprises Vitamin K2 in an amount between about 75 micrograms and about 100 micrograms per dose or serving of the composition. As discussed above, the Vitamin K2 may be in the form of MK-4, MK-7, or a combination thereof. An example of a suitable commercially available source of Vitamin K2 (as MK-7) is MenaQ7 available from NattoPharma ASA (Høvik, Norway).

### Macronutrients

As previously discussed, in certain exemplary embodiments, the composition comprising Curcumin and Vitamin K2 may be formulated as a nutritional composition. In one exemplary embodiment, the nutritional composition may further comprise one or more macronutrients. In one exemplary embodiment, the nutritional composition further comprises at least one source of protein. In one exemplary embodiment, the nutritional composition further comprises at least one source of carbohydrate. In one exemplary embodiment, the nutritional composition further comprises at least one source of fat. In one exemplary embodiment, the composition comprising Curcumin and Vitamin K2 further comprises at least one source of protein, at least one source of carbohydrate, and at least one source of fat. In one exemplary embodiment, the composition comprising Curcumin and Vitamin K2 further comprises at least one source of protein, at least one source of carbohydrate, and at least one source of fat to provide a complete nutritional composition (i.e., the composition contains sufficient types and levels of macronutrients (protein, carbohydrate, and fat) and micronutrients sufficient to provide a sole source of nutrition for the individual to which it is administered).

Macronutrients suitable for use in the exemplary methods and compositions described herein include any protein, carbohydrate, or fat or source thereof, which is now known to be or becomes known in the future to be, suitable for use in an oral nutritional composition, provided that the macronutrient is safe and effective for oral administration and is otherwise compatible with the other ingredients in the nutritional composition.

Generally, the concentration or amount of protein, carbohydrate, or fat in the nutritional composition can vary considerably depending upon the particular product form (e.g., bars or other solid dosage forms, milk or soy-based liquids/emulsions, clear beverages, reconstitutable powders) and targeted dietary needs. For the exemplary embodiments disclosed herein, these macronutrients will often be formulated within any of the exemplary ranges described in Tables 1 and 2.

TABLE 1

| Nutrient (% total calories) | Example A | Example B | Example C |
|---|---|---|---|
| Carbohydrate | 0-100 | 10-70 | 40-50 |
| Fat | 0-100 | 20-65 | 35-55 |
| Protein | 0-100 | 5-40 | 15-25 |

Each numerical value preceded by the term “about.”

TABLE 2

| Nutrient (wt % composition) | Example D | Example E | Example F |
|---|---|---|---|
| Carbohydrate | 0-98 | 1-50 | 10-30 |
| Fat | 0-98 | 1-30 | 3-15 |
| Protein | 0-98 | 1-30 | 2-10 |

Each numerical value preceded by the term “about.”

In one exemplary embodiment, the nutritional composition comprises at least one source of protein. In one exemplary embodiment, the nutritional composition comprises at least one source of protein in an amount sufficient to provide about 5 grams to about 50 grams of protein per serving of the nutritional composition. In certain exemplary embodiments, the nutritional composition comprises about 5 grams to about 40 grams, about 10 grams to about 35 grams, or about 15 grams to about 30 grams of protein per serving. In one exemplary embodiment, the composition comprises about 1% to about 30% of at least one source of protein by weight of the composition. In one exemplary embodiment, the composition comprises about 1% to about 20% of at least one source of protein by weight of the composition. In one exemplary embodiment, the composition comprises about 1% to about 15%, about 1% to about 10%, about 1% to about 7%, or about 1% to about 5% of at least one source of protein by weight of the composition. Any source of protein may typically be used in the nutritional compositions so long as it is suitable for oral administration and is otherwise compatible with any other selected ingredients or features in the nutritional composition.

The at least one source of protein may include, but is not limited to, intact, hydrolyzed, and partially hydrolyzed protein, which may be derived from any known or otherwise suitable source such as milk (e.g., casein, whey), animal (e.g., meat, fish), cereal (e.g., rice, corn, wheat), vegetable (e.g., soy, potato, pea), and combinations thereof. Non-limiting examples of the at least one source of protein include whey protein concentrates, whey protein isolates, whey protein hydrolysates, acid caseins, sodium caseinates, calcium caseinates, potassium caseinates, casein hydrolysates, milk protein concentrates, milk protein isolates, milk protein hydrolysates, nonfat dry milk, condensed skim milk, soy protein concentrates, soy protein isolates, soy protein hydrolysates, pea protein concentrates, pea protein isolates, pea protein hydrolysates, insect proteins, earthworm proteins, potato protein, rice protein, corn protein, wheat protein, sunflower protein, chickpea protein, quinoa protein, and combinations thereof. In addition, the at least one source of protein may comprise any one source of protein or any combination of two or more distinct sources of protein, such as any of the various sources of protein provided in the non-limiting list presented above.

In one exemplary embodiment, the at least one source of protein may also include, or be entirely or partially replaced by, free amino acids, non-limiting examples of which include L-tryptophan, L-glutamine, L-tyrosine, L-methionine, L-cysteine, taurine, L-arginine, L-carnitine, and combinations thereof.

In one exemplary embodiment, the nutritional composition comprises at least one source of carbohydrate. In certain exemplary embodiments, the nutritional composition comprises about 15 grams to about 110 grams of at least one source of carbohydrate per serving. In other exemplary embodiments, the nutritional composition comprises about 25 grams to about 90 grams, including about 40 grams to about 65 grams, and also including about 45 grams to about 55 grams of at least one source of carbohydrate per serving. In one exemplary embodiment, the composition comprises about 10% to about 80% of at least one source of carbohydrate by weight of the composition. In certain exemplary embodiments, the composition comprises about 20% to about 70%, about 30% to about 65%, or about 40% to about 60% of at least one source of carbohydrate by weight of the composition.

The at least one source of carbohydrate may be simple, complex, or variations or combinations thereof. Generally, any source of carbohydrate may be used so long as it is suitable for oral administration and is otherwise compatible with any other selected ingredients or features present in the nutritional composition. Non-limiting examples of a source of carbohydrate suitable for use in the exemplary methods and compositions described herein include hydrolyzed or modified or resistant starch or cornstarch, maltodextrin, isomaltulose, sucromalt, glucose polymers, sucrose, corn syrup, corn syrup solids, rice-derived carbohydrate, glucose, fructose, lactose, high fructose corn syrup, honey, sugar alcohols (e.g., maltitol, erythritol, sorbitol), and combinations thereof.

In one exemplary embodiment, the at least one source of carbohydrate may comprise one or more of soluble dietary fiber and insoluble dietary fiber. Examples of suitable soluble dietary fiber for use in the exemplary methods and compositions described herein include, but are not limited to, gum arabic, sodium carboxymethyl cellulose, guar gum, citrus pectin, low and high methoxy pectin, oat and barley glucans, carrageenan, psyllium and combinations thereof. Examples of suitable insoluble dietary fiber for use in the exemplary methods and compositions described herein include, but are not limited to, oat hull fiber, pea hull fiber, soy hull fiber, soy cotyledon fiber, sugar beet fiber, cellulose, corn bran, and combinations thereof.

In one exemplary embodiment, the nutritional composition comprises at least one source of fat. In certain exemplary embodiments, the nutritional composition comprises no fat, or essentially no fat (i.e., less than 0.5 grams of fat per serving). In one exemplary embodiment, the nutritional composition comprises about 0.5 grams to about 45 grams of at least one source of fat per serving. In other exemplary embodiments, the nutritional composition comprises about 2 grams to about 35 grams, about 5 grams to about 30 grams, about 10 grams to about 25 grams, or about 15 grams to about 20 grams of at least one source of fat per serving. In one exemplary embodiment, the composition comprises about 0.5% to about 30% of at least one source of fat by weight of the composition. In certain exemplary embodiments, the composition comprises about 1% to about 30%, about 5% to about 25%, about 10% to about 20%, or about 12% to about 18% of at least one source of fat by weight of the composition. In certain exemplary embodiments, the composition comprises about 1% to about 18%, about 1.5% to about 10%, or about 2% to about 5% of at least one source of fat by weight of the composition.

In general, any source of fat may be used so long as it is suitable for oral administration and is otherwise compatible with any other selected ingredients or features present in the exemplary compositions described herein. The at least one source of fat may be derived from plants, animals, and combinations thereof. Non-limiting examples of suitable sources of fat for use in the exemplary compositions described herein include coconut oil, fractionated coconut oil, soy oil, corn oil, olive oil, safflower oil, high oleic safflower oil, high GLA-safflower oil, MCT (medium chain triglycerides) oil, sunflower oil, high oleic sunflower oil, palm oil, palm kernel oil, palm olein, canola oil, marine (e.g., tuna, sardine) oil, flaxseed oil, borage oil, cottonseed oil, evening primrose oil, blackcurrant seed oil, transgenic oil sources, fungal oils, and combinations thereof.

### Calcium

Calcium is an important component of a healthy diet and a mineral necessary for life. Approximately ninety-nine percent of the body's calcium is stored in the bones and teeth. The remaining calcium in the body has other important uses, such as exocytosis, especially neurotransmitter release, and muscle contraction. Calcium deficiency, particularly in the case of menopausal or postmenopausal women, can lead to osteoporosis, in which the bone deteriorates and there is an increased risk of fractures.

In one exemplary embodiment, the composition, including a nutritional composition based thereon, further comprises calcium, or a source of calcium. In certain exemplary embodiments, the composition includes a source of calcium in an amount sufficient to provide about 150 milligrams to about 800 milligrams of calcium (as elemental calcium) per serving or dose. In certain exemplary embodiments, the composition includes an amount of calcium between about 200 milligrams and about 600 milligrams, between about 250 milligrams and about 500 milligrams, or between about 300 milligrams and about 400 milligrams per serving or dose. Exemplary sources of calcium suitable for use in the compositions described herein include, but are not limited to, calcium carbonate, calcium caseinate, calcium citrate, calcium chloride, calcium lactate, calcium acetate, and calcium aspartate.

### Vitamin D3

Vitamin D3 is a compound that is naturally produced in the skin in response to sunlight, and is also present in certain foodstuffs (particularly oily fish). Vitamin D3 is a type of steroid hormone and among other things, a powerful mediator of immune function. In addition, Vitamin D3 is well known for its effect on calcium metabolism. Proper levels of Vitamin D3 are necessary to maintain bone mineral density and serum (blood) calcium levels.

In one exemplary embodiment, the composition, including a nutritional composition based thereon, comprises Vitamin D3 (cholecalciferol). In one exemplary embodiment, the composition comprises about 160 IU (4 micrograms) to about 1,000 IU (25 micrograms) of Vitamin D3 per serving or dose. In certain exemplary embodiments, the composition comprises about 400 IU (10 micrograms) to about 800 IU (20 micrograms), about 400 IU (10 micrograms) to about 600 IU (15 micrograms), or about 160 IU (4 micrograms) to about 240 IU (6 micrograms) per serving or dose. An example of a suitable commercially available source of Vitamin D3 is Qali-D (Vitamin D) available from DSM (Netherlands).

### Optional Ingredients

In certain exemplary embodiments, the nutritional composition may comprise other optional ingredients, for example, to modify the physical, chemical, aesthetic, or processing characteristics of the nutritional composition, or to provide additional nutritional benefits. Many such optional ingredients are known to be suitable for use in nutritional products and may also be used in the nutritional compositions described herein, provided that such optional ingredients are safe for oral administration and are compatible with the essential and other ingredients in the selected product form.

Non-limiting examples of such other optional ingredients include preservatives, anti-oxidants, buffers, pharmaceutical actives, sweeteners, colorants, flavors, flavor enhancers, thickening agents and stabilizers, emulsifying agents, prebiotics, probiotics, anti-inflammatory agents, lubricants, and combinations thereof.

In one exemplary embodiment, the nutritional composition may comprise at least one vitamin (in addition to Vitamin K2), at least one mineral, and combinations thereof. Exemplary vitamins that may be used in the nutritional composition include, but are not limited to, Vitamin A, Vitamin B12, Vitamin C, Vitamin D2, Vitamin D3, Vitamin E, Vitamin K1, Vitamin A palmitate, Vitamin C palmitate (ascorbyl palmitate), Vitamin E acetate, thiamine, riboflavin, pyridoxine, carotenoids (e.g., beta-carotene, zeaxanthin, lutein, lycopene), niacin, folic acid, pantothenic acid, biotin, choline, inositol, and various salts, esters, or other derivatives thereof, and combinations thereof. Exemplary minerals that may be used in the nutritional composition include, but are not limited to, calcium, selenium, potassium, iodine, phosphorus, magnesium, iron, zinc, manganese, copper, sodium, molybdenum, chromium, chloride, and combinations thereof.

In one exemplary embodiment, the nutritional composition may comprise at least one sweetening agent. In certain exemplary embodiments, the at least one sweetening agent is a sugar alcohol such as maltitol, erythritol, sorbitol, xylitol, mannitol, isomalt, and lactitol, or at least one artificial or high potency sweetener such as acesulfame K, aspartame, sucralose, saccharin, stevia, and tagatose, and combinations thereof. The sweetening agents, especially as a combination of a sugar alcohol and an artificial sweetener, can be useful in formulating liquid nutritional compositions having a desirable favor profile. These sweetener combinations can also be effective in masking undesirable flavors, for example, as sometimes associated with the addition of vegetable proteins to a liquid nutritional composition.

In one exemplary embodiment, the nutritional composition may comprise a flowing agent or anti-caking agent to retard clumping or caking of a nutritional powder embodiment over time and to make the nutritional powder flow easily from its container. Any flowing or anti-caking agents that are known or otherwise suitable for use in a nutritional powder or product form may be suitable for use herein, non-limiting examples of which include tricalcium phosphate, silicates, and combinations thereof. The concentration of the flowing agent or anti-caking agent will often vary depending upon the product form, the other selected ingredients, the desired flow properties, and so forth.

In one exemplary embodiment, the nutritional composition may comprise a stabilizer. Any stabilizer that is known or otherwise suitable for use in a nutritional composition may also be suitable for use herein, non-limiting examples of which include gums such as xanthan gum and locust bean gum.

In certain exemplary embodiments, the nutritional composition optionally includes one or more masking agents to reduce or otherwise obscure the development of any residual bitter flavors and after taste in the nutritional composition over time. Suitable masking agents include natural and artificial sweeteners, sodium sources such as sodium chloride, and hydrocolloids such as guar gum, xanthan gum, carrageenan, gellan gum, and combinations thereof. The amount of masking agent used will often vary depending upon the particular masking agent selected, other ingredients in the formulation, and other formulation or product target variables.

### Methods of Manufacture

The exemplary nutritional compositions may be prepared by any process or method (now known or known in the future) suitable for making a selected product form, such as a nutritional solid, a nutritional powder, or a nutritional liquid. Many such techniques may be known for any given product form, such as nutritional liquids or nutritional powders, and can readily be applied by one of ordinary skill in the art to the various exemplary embodiments described herein.

In one suitable manufacturing process, a nutritional liquid is prepared using at least three separate slurries, including a protein-in-fat (PIF) slurry, a carbohydrate-mineral (CHO-MIN) slurry, and a protein-in-water (PIW) slurry. The PIF slurry is formed by heating and mixing selected oils (e.g., canola oil, corn oil, fish oil) and then adding an emulsifier (e.g., soy lecithin), fat soluble vitamins (e.g., Vitamin K2), and a portion of the total protein (e.g., milk protein concentrate) with continued heat and agitation. The CHO-MN slurry is formed by adding with heated agitation to water: Curcumin, minerals (e.g., potassium citrate, dipotassium phosphate, sodium citrate), trace minerals and ultra trace minerals (e.g., TM/UTM premix), thickening or suspending agents (e.g., gellan gum, carrageenan). The resulting CHO-MIN slurry is held for 10 minutes with continued heat and agitation before adding additional minerals (e.g., potassium chloride, magnesium carbonate, potassium iodide), and carbohydrates (e.g., sucrose, corn syrup). The PIW slurry is then formed by mixing with heat and agitation the remaining protein (e.g., sodium caseinate, soy protein concentrate) into water.

The resulting slurries are then blended together with heated agitation and the pH adjusted to a desired range, typically 6.6-7.0, after which the composition is subjected to high-temperature short-time (HTST) processing during which the composition is heat treated, emulsified and homogenized, and then allowed to cool. Water soluble vitamins and ascorbic acid are added, the pH is again adjusted to the desired range (if necessary), flavors are added, and water is added to achieve a desired total solid level. The composition is then aseptically packaged to form an aseptically packaged nutritional emulsion, or the composition is added to retort stable containers and then subjected to retort sterilization to form retort sterilized nutritional emulsions.

The manufacturing processes for the nutritional emulsions may be carried out in ways other than those set forth herein without departing from the spirit and scope of the general inventive concepts. The disclosed embodiments are, therefore, to be considered in all respects illustrative and non-restrictive with changes and equivalents intended to be encompassed therein in accordance with the general inventive concepts.

A nutritional powder, such as a spray dried nutritional powder, may be prepared by any combination of known or otherwise effective techniques suitable for making and formulating a spray dried nutritional powder. The spray drying step may likewise include any spray drying technique that is known for or otherwise suitable for use in the production of nutritional powders. Many different spray drying methods and techniques are known for use in the nutrition field, each of which may be suitable for use in the manufacture of the exemplary nutritional powders described herein.

One method of preparing an exemplary spray dried nutritional powder comprises forming and homogenizing an aqueous slurry or liquid comprising Curcumin, Vitamin K2, and at least one source of protein, and then spray drying the slurry or liquid to produce a spray dried nutritional powder. The method may further comprise the step of spray drying, dry mixing, or otherwise adding additional nutritional ingredients, including any one or more of the ingredients described herein, to the spray dried nutritional powder. In certain exemplary embodiments, the methods of manufacture may utilize Curcumin formulated as bioavailable Curcumin and menaquinone-7.

### Methods of Use

The exemplary compositions comprising a combination of Curcumin and Vitamin K2 may be used to improve one or more of bone health, bone strength, and joint health. Particularly, it has been surprisingly found that the combination of Curcumin and Vitamin K2 synergistically inhibits osteoclast differentiation, and consequently inhibits collagen degradation, to thereby improve bone health and joint health. Further, the combination of Curcumin and Vitamin K2 suppresses bone resorption. These benefits are important in maintaining the integrity of bones as well as joints, which provide protection against the pathogenesis of osteoarthritis and other conditions resulting in bone damage or loss including, but not limited to, systemic lupus erythematosus (SLE), post-menopausal osteoporosis, corticosteroid treatment, anorexia, disuse from stroke, and Parkinson's Disease. Accordingly, the exemplary compositions comprising a combination of Curcumin and Vitamin K2 describe herein may benefit individuals by preventing, controlling, reducing, or treating occurrences of conditions that result in reduced bone health, joint health, or bone loss. Any of the previously described exemplary compositions may be used in the exemplary methods described herein.

The exemplary compositions comprising a combination of Curcumin and Vitamin K2 may provide anti-inflammatory benefits, which is important since inflammation contributes significantly to the pathogenesis of a number of bone and joint conditions, such as osteoarthritis, rheumatoid arthritis, SLE, osteopenia, or osteoporosis. Therefore, the exemplary methods and compositions described herein may further prevent, control, reduce, or treat osteoarthritis, rheumatoid arthritis, SLE, osteopenia, or osteoporosis.

In one exemplary embodiment, a method of administering a composition comprising a combination of Curcumin and Vitamin K2 may be used to treat low-grade inflammation in an individual in need thereof. Low-grade inflammation has been associated with joint degradation, and may be a trigger that causes autoimmune responses that deteriorate synovial joints. By functionally addressing an underlying cause of joint deterioration, the exemplary methods and compositions described herein may prevent or significantly delay joint deterioration, particularly age-related joint deterioration and progression to osteoarthritis.

In one exemplary embodiment, a method of administering a composition comprising a combination of Curcumin and Vitamin K2 may be used to inhibit osteoclast differentiation in an individual in need thereof. By inhibiting osteoclast differentiation, osteoclastic bone resorption is inhibited or significantly reduced, which ultimately lessens bone loss. The reduced bone loss can also serve to maintain the integrity of bone at that joints, known as subchondral bone, the erosion of which contributes to the pathogenesis of joint disease, such as osteoarthritis.

In one exemplary embodiment, a method of administering a composition comprising Curcumin and Vitamin K2 to an individual in need thereof may be used to improve or maintain bone health, joint health, or both. In one exemplary embodiment, the individual in need thereof is a human having or diagnosed as at risk for one or more of osteoarthritis, rheumatoid arthritis, SLE, osteopenia, and osteoporosis. In one exemplary embodiment, the individual in need thereof is a menopausal or post-menopausal woman. In general, the menopausal or post-menopausal woman is estrogen deficient, which can cause osteoporosis and a corresponding reduction in bone mineral density. In one exemplary embodiment, the individual in need thereof has degenerated cartilage in one or more joints, including, but not limited to, knee joints, hip joints, shoulder joints, elbows, and wrists. In one exemplary embodiment, the individual in need thereof is diagnosed as having, or exhibiting symptoms associated with, low-grade inflammation.

In one exemplary embodiment, an individual in need thereof may refer to a subset of individuals in need of improved bone health, joint health, or both. In one exemplary embodiment, such a subset of individuals in specific need of improved bone health, joint health, or both may include infants, pediatrics, teens, or adults who experience, are susceptible to, or are at elevated risk of experiencing osteoarthritis, rheumatoid arthritis, osteoporosis, or fragile bones. In one exemplary embodiment, such a subset of individuals in specific need of improved bone health, joint health, or both may include infants, pediatrics, teens, or adults who experience, are susceptible to, or are at elevated risk of systemic lupus erythematosus (SLE), post-menopausal osteoporosis, corticosteroid treatment, anorexia, disuse from stroke and Parkinson's Disease, and the like. Preterm infants, infants, pediatrics, teens, adults, and older adults may be susceptible to or at elevated risk for experiencing these diseases and conditions due to one or more of family history, age, environment, and lifestyle. Based on the foregoing, because some of the exemplary methods described herein are directed to specific subsets or subclasses of identified individuals (that is, the subset or subclass of individuals “in need” of assistance in addressing one or more specific conditions noted herein), not all individuals will fall within the subset or subclass of individuals as described herein for certain diseases or conditions.

In one exemplary embodiment, an exemplary composition comprising Curcumin and Vitamin K2 may be administered to an individual in need thereof one or more times per day for a period suitable to achieve the desired effect. For example, according to one exemplary embodiment, an exemplary composition is administered to an individual in need thereof one a day for at least a week, once a day for at least two weeks, once a day for at least a month, once a day for at least 6 months, or once a day for a year or more. In certain other exemplary embodiments, an exemplary composition is administered to an individual in need thereof multiple (e.g., two) times a day for at least a week, multiple (e.g., two) times a day for at least two weeks, multiple (e.g., two) times a day for at least a month, multiple (e.g., two) times a day for at least 6 months, or multiple (e.g., two) times a day for a year or more. Within the context of administering a dose or serving of an exemplary composition to an individual in need thereof, according to one exemplary embodiment, every day is intended to reflect an individual who has been instructed to be administered the composition daily and who actually is administered the composition for at least 70% (and in certain other exemplary embodiments at least 90%) of the days during the period of administration.

In one exemplary embodiment, an exemplary composition comprising Curcumin and Vitamin K2 is acutely administered to the individual in need thereof. The phrases “acutely administered” and “acute administration,” as used herein, refer to administration of the exemplary composition to the individual in need thereof on a non-regular basis. Acute administration may be a single serving, or multiple servings, administered over a relatively short time period, such as up to three weeks, including one day, two days, three days, five days, one week, ten days, two weeks, or three weeks.

In one exemplary embodiment, an exemplary composition comprising Curcumin and Vitamin K2 is chronically administered to the individual in need thereof. The phrases “chronically administered” and “chronic administration,” as used herein, refers to regular administration of the exemplary composition to the individual in need thereof on an indefinite regular basis, or to regular administration of the exemplary composition to the individual in need thereof for a significant period of time. For example, in certain exemplary embodiments, chronic administration can include regular administration for at least three weeks, regular administration for at least one month, regular administration for at least 6 weeks, regular administration for at least two months, regular administration for at least 3 months, regular administration for at least 4 months, regular administration for at least 5 months, regular administration for at least 6 months, or regular administration for at least 9 months. In other exemplary embodiments, chronic administration refers to regular administration for at least 1 year, regular administration for at least 1.5 years, regular administration for at least 2 years, or regular administration for more than 2 years. “Regular administration,” as used herein, refers to administration according to a schedule whereby the individual in need thereof will receive the exemplary composition at regular intervals.

As used herein, “regular intervals” refers to administration in a repeating, periodic fashion where the time between administrations is approximately (or intended to be approximately) the same. In various exemplary embodiments, administration at regular intervals includes daily administration or weekly administration. In other exemplary embodiments, administration at regular intervals includes administration 1-2 times per week, administration 1-3 times per week, administration 2-3 times per week, administration 1-4 times per week, administration 1-5 times per week, administration 2-5 times per week, administration 3-5 times per week, administration 1-6 times per week, administration 1-7 times per week, administration 2-6 times per week, administration 2-7 times per week, administration 1-2 times per day, administration 1-3 times per day, administration 1-4 times per day, administration 2-3 times per day, administration 2-4 times per day, administration 3-4 times per day, administration 2-5 times per day, administration 3-5 times per day, or administration 4-5 times per day.

In one exemplary embodiment, a nutritional composition comprising at least one source of protein in an amount sufficient to provide about 5 grams to about 50 grams of protein per serving, Curcumin, Vitamin K2, Vitamin D3, and calcium is administered to an individual in need thereof. Upon consumption of the nutritional composition, the bone quality of the individual in need thereof is maintained. In certain exemplary embodiments, the nutritional composition may comprise effective amounts of each of Curcumin, Vitamin K2, Vitamin D3, and calcium. For example, the nutritional composition may comprise, per dose or serving: Curcumin in an amount between about 1 milligram and about 10,000 milligrams, Vitamin K2 in an amount between about 25 micrograms and about 200 micrograms, Vitamin D3 in an amount between about 4 micrograms and about 25 micrograms, and calcium in an amount between about 150 milligrams to about 800 milligrams.

In one exemplary embodiment, a nutritional composition comprising at least one source of protein in an amount sufficient to provide 5 grams to 50 grams of protein per serving, Curcumin, Vitamin K2, Vitamin D3, and calcium is administered to an individual having or diagnosed as at risk for bone loss. Upon consumption of the nutritional composition, the bone loss of the individual is reduced. In certain exemplary embodiments, the nutritional composition may comprise effective amounts of each of Curcumin, Vitamin K2, Vitamin D3, and calcium. For example, the nutritional composition may comprise, per dose or serving: Curcumin in an amount between about 1 milligram and about 10,000 milligrams, Vitamin K2 in an amount between about 25 micrograms and about 200 micrograms, Vitamin D3 in an amount between about 4 micrograms and about 25 micrograms, and calcium in an amount between about 150 milligrams to about 800 milligrams.

The terms “maintain,” “maintained,” or “maintenance” when used herein in connection with bone quality, or a characteristic of bone quality, refer to retaining an amount of bone quality that corresponds to the bone quality of an individual prior to initiating the methods disclosed herein, or a percentage thereof, or even an increase in bone quality, or a characteristic of bone quality. As previously mentioned, bone quality is indicated by various characteristics including, but not limited to, bone mineral density (BMD), bone strength, bone mineral content (BMC), bone turnover, and bone microarchitecture.

A variety of techniques may be used to determine, either quantitatively or qualitatively, the characteristics that provide an indication of bone quality. For example, a dual energy X-Ray absorptiometry (DXA) scan may be used to measure BMC and BMD. BMC determines the mass of mineral present in the whole body or in a selected bone region. BMC changes reflect the result of the metabolic “mass” balance between bone formation and bone destruction. BMC represented relative to the projected bone area refers to BMD. BMD represents the whole mass of mineral present in the bone region studied. Other techniques for measuring BMC and BMD include single-photon absorptiometry, dual-photon absorptiometry, and quantitative computed tomography.

Another useful technique for providing an indication of bone quality is micro-computerized tomography (micro-CT). A micro-CT scan allows for the nondestructive assessment and analysis of bone microarchitecture (e.g., three-dimensional trabecular and cortical bone structural properties). Scanning with micro-CT can be achieved at resolutions as low as 5 μm, allowing for the determination of porosities and subtle modeling and remodeling events of the bone tissue. Furthermore, true three-dimensional image reconstructions permit the assessment of bone microarchitecture as a three-dimensional structure, providing critical information to images collected through histomorphometry.

Bone turnover can be assessed, for example, by measurements of biochemical markers. Serum alkaline phosphatase, osteocalcin, and procollagen type I propeptides can be used as indices of bone formation, while urinary hydroxyproline, pyridinoline and deoxypyridinoline can be used to assess bone resorption.

In one exemplary embodiment, the bone quality that is maintained is BMD, bone strength, or combinations thereof. In one exemplary embodiment, where the individual in need thereof consumes the nutritional composition comprising at least one source of protein in an amount sufficient to provide about 5 grams to about 50 grams of protein per serving, Curcumin, Vitamin K2, Vitamin D3, and calcium, the level of maintenance of bone quality is about 75% to about 130% of the BMD of the individual prior to initiating the exemplary methods described herein. In certain other exemplary embodiments, where the individual in need thereof consumes the nutritional composition comprising at least one source of protein in an amount sufficient to provide about 5 grams to about 50 grams of protein per serving, Curcumin, Vitamin K2, Vitamin D3, and calcium, the level of maintenance of bone quality is about 80% to about 130%, about 90% to about 130%, about 100% to about 130%, about 110% to about 130%, or about 120% to about 130% of the BMD of the individual prior to initiating the exemplary methods described herein. As previously discussed, the BMD of the individual may be determined by various clinical techniques, such as DXA.

According to one exemplary embodiment, a method of administering a nutritional composition comprising at least one source of protein in an amount sufficient to provide 5 grams to 50 grams of protein per serving, Curcumin, Vitamin K2, Vitamin D3, and calcium to an individual in need thereof may be used to reduce bone loss in the individual. Reduction of bone loss may also be considered as maintenance of BMD. In one exemplary embodiment, the reduction in bone loss is an increase in BMD. In accordance with one exemplary embodiment, where the individual in need thereof consumes the nutritional composition comprising at least one source of protein in an amount sufficient to provide 5 grams to 50 grams of protein per serving, Curcumin, Vitamin K2, Vitamin D3, and calcium, the reduction of bone loss (based upon maintenance of BMD) is from 75% to 130% of the BMD of the individual prior to initiating the exemplary methods described herein. In certain other exemplary embodiments, where the individual in need thereof consumes the nutritional composition comprising at least one source of protein in an amount sufficient to provide about 5 grams to about 50 grams of protein per serving, Curcumin, Vitamin K2, Vitamin D3, and calcium, the level of maintenance of bone quality is about 80% to about 130%, about 90% to about 130%, about 100% to about 130%, about 110% to about 130%, or about 120% to about 130% of the BMD of the individual prior to initiating the exemplary methods described herein. In the case where the reduction of bone loss is 100% or more, consumption of the nutritional composition is effective to prevent bone loss in the subject. As previously discussed, the BMD of the subject may be determined by various clinical techniques, including DXA.

In terms of measuring the maintenance of bone quality or the reduction of bone loss, a first measurement of the bone quality or bone loss (e.g., BMD) of the subject may be performed prior to initiating the exemplary methods described herein. In one exemplary embodiment, the first measurement is performed a week (e.g., 1-7 days) before initiation of the exemplary methods described herein. Next, a second measurement of the bone quality or bone loss of the subject is performed at some time point after initiating the methods disclosed herein, and the second measurement is compared to the first measurement. Notably, the comparison of the second measurement to the first measurement may not show immediate results using the aforementioned measurement techniques. The resulting effect may take days, weeks, or months of regular administration of an exemplary composition described herein according to the dosages and in the intervals previously described herein to obtain the stated measurable results described above. In one exemplary embodiment, the amount of time between the first measurement of bone quality or bone loss and the second measurement of bone quality or bone loss is two weeks, one month, two months, six months, or more. In one exemplary embodiment, for the purpose of determining the effects of the exemplary methods described herein, a 3-12 month test period of regular administration of an exemplary composition may be used. In one exemplary embodiment, for the purpose of determining the effects of the exemplary methods described herein, a 2 week to 3 month test period of regular administration of an exemplary composition may be used.

Accordingly, administration of an exemplary composition described herein may be effective for improving or maintaining bone health, joint health, or both in an individual in need thereof. Additionally, administration of an exemplary composition described herein may be effective for reducing bone loss, maintaining bone quality, or both in an individual in need thereof. While not wishing to be bound by any particular theory, it is believed that the combination of Curcumin and Vitamin K2 act synergistically to increase intestinal calcium absorption, promote bone mineralization by osteoblasts, and inhibit osteoclast differentiation and activity. Furthermore, it is believed that the combination of Curcumin and Vitamin K2 act synergistically to inhibit collagen degradation and suppress bone resorption to thereby improve bone health and joint health. Several exemplary methods and compositions are further described herein in the following examples.

### EXAMPLES

The following examples illustrate exemplary embodiments of methods and compositions encompassed by the general inventive concepts. The examples are given solely for the purpose of illustration and are not to be construed as limitations of the general inventive concepts, as many variations thereof are possible without departing from the spirit and scope of the general inventive concepts described herein. All exemplified amounts are weight percentages based upon the total weight of the product, unless otherwise specified.

### Example 1

In this Example, the effects of Curcumin and Vitamin K2, alone and in combination, on osteoclast differentiation and collagen degradation in vitro were evaluated.

A 96-well OsteoLyse™ assay system (commercially available from Lonza Biosciences, Walkersville, Md.) was used for this evaluation. Specifically, a 96-well OsteoLyse™ plate was coated with fluorophore-derivatized human bone matrix (europium conjugated collagen). Human osteoclast precursors, at a density of 10,000/200 μl of differentiation medium (containing M-CSF and RANK Ligand) were seeded onto the surface of the plate. Triplicate cell culture wells were treated with one of: Curcumin in DMSO; Vitamin K2 in ethanol; combination of Curcumin and Vitamin K2; and alendronate (control). Cell culture wells with only differentiation medium were treated as standard control wells.

After six days of culture, old medium was removed and 200 μl of fresh differentiation medium was added to all wells. Twenty-four hours later, on day seven of culture, 10 μl of medium supernatant was used for fluorescence measurement. For the measurement, 10 μl of medium supernatant was added to 100 μl of fluorophore-releasing agent (Lonza Bioscience, Walkersville, Md.) in a black-walled 96-well plate.

The samples were briefly mixed and fluorescence was determined using a time-resolved fluorescence fluorimeter (Spectramax M5, available from Molecular Devices, Sunnyvale, Calif., with excitation at 340 nm and emission at 615 nm) over a 400-microsecond time period after an initial delay of 400 microseconds.

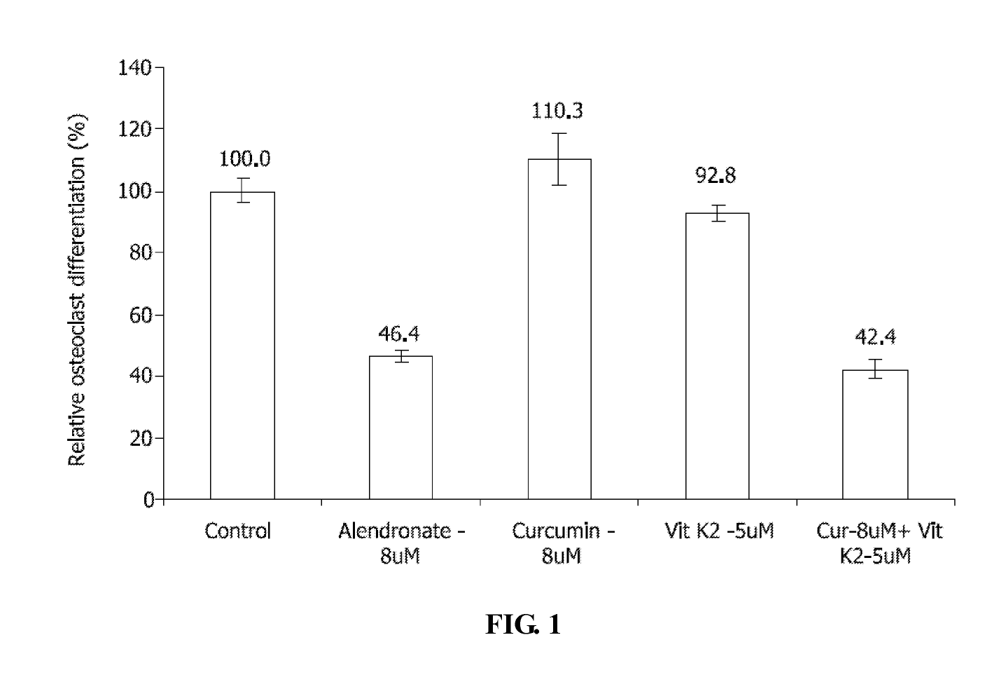

Osteoclast differentiation in a standard control cell culture well was assumed to be 100% and relative osteoclast differentiation, with respect to control wells, in other cell culture wells was calculated and plotted on the bar graph shown in FIG. 1.

As shown in FIG. 1, the combination of Curcumin and Vitamin K2 synergistically inhibited osteoclast differentiation by 57.6%, similar to the inhibition by positive control, Alendronate (53.6%). Curcumin and Vitamin K2 individually, however, did not significantly inhibit osteoclast differentiation.

### Example 2

In this Example, the effects of Curcumin and Vitamin K2, alone and in combination, on bone resorption was evaluated.

A 96-well OsteoAssay™ plate (commercially available from Lonza Biosciences, Walkersville, Md.) was used for this evaluation. Specially, a 96-well OsteoAssay™ plate was coated with a thin-layer of adherent human bone particles. Primary human osteoclast precursors, at a density of 10,000/200 μl of differentiation medium (containing M-CSF and RANK Ligand), were seeded onto the surface of the plate. Triplicate cell culture wells were treated with one of: Curcumin in DMSO; Vitamin K2 in ethanol; combination of Curcumin and Vitamin K2; and alendronate (control). Cell culture wells with only differentiation medium were treated as standard control wells.

After six days of culture, old medium was removed and 200 μl of fresh basal resorption medium containing M-CSF and RANK Ligand was added to all wells. Forty-eight hours later, on day eight of culture, 10 μl of medium supernatant was used for calcifluor assay. For the assay, CalciFluor™ Assay kit (commercially available from Lonza Biosciences, Walkersville, Md.) was used. The kit measures the calcium that is released as a result of osteoclast-medium resorptive activity. The appearance of free calcium in cell culture medium is a direct result of cell-mediated bone resorption. One vial of calcium detection reagent (Lonza Bioscience, Walkersville, Md.) was diluted in 200 ml of detection reagent diluent (Lonza Bioscience, Walkersville, Md.) and left to equilibrate for 15 minutes.

10 μl of cell culture medium supernatant was added to 200 μl of reconstituted calcium detection reagent in a black-walled 96-well plate. In addition, serial dilutions of a calcium standard were prepared and 10 μl of the standard was added to 200 μl of calcium detection reagent, to prepare the standard curve for calcium. The samples were briefly mixed and fluorescence was determined using a fluorimeter (Spectramax M5, available from Molecular Devices, Sunnyvale, Calif., with excitation at 551 nm and emission at 577 nm).

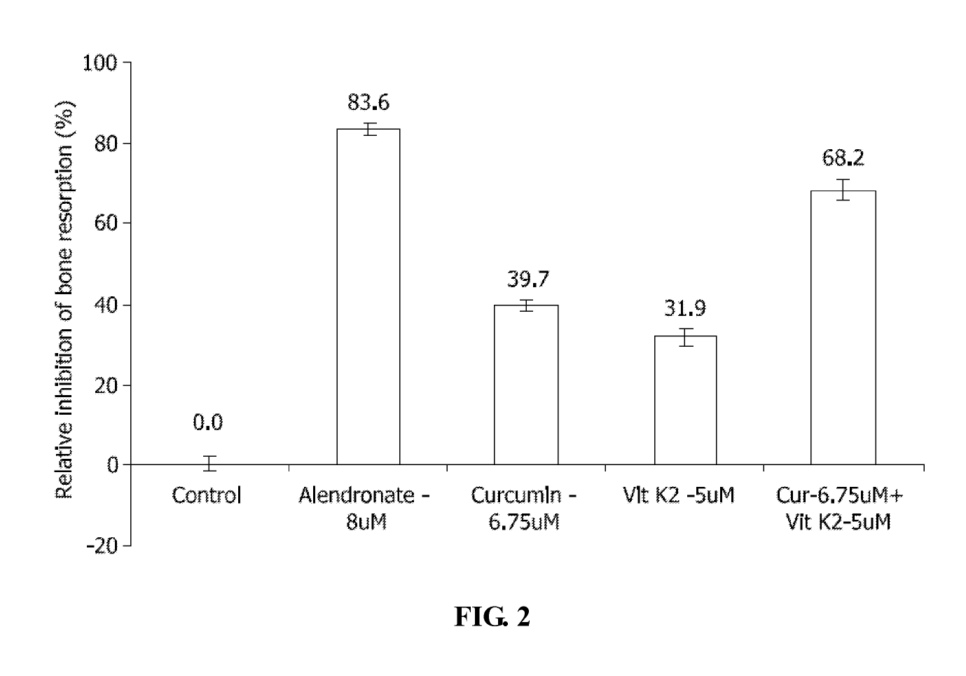

The concentration of calcium in each well was calculated based on the standard curve. The concentration of calcium is directly proportional to bone resorption. The relative percentage inhibition of bone resorption, with respect to standard control cells, was calculated and plotted on the bar graph as shown in FIG. 2.

As shown in FIG. 2, Curcumin and Vitamin K2 individually inhibited bone resorption, 39.7% and 31.9%, respectfully. Moreover, the combination of Curcumin and Vitamin K2 significantly inhibited bone resorption by 68.2%.

### Example 3

Example 3 illustrates an exemplary composition formulated as a nutritional powder, the ingredients of which are listed in Table 3 below. All ingredient amounts are listed as kg per 1000 kg batch of product, unless otherwise specified.

TABLE 3

| Ingredient | Example 3 |
|---|---|
| Skim Milk Powder | 502.9 |
| Corn Syrup Solids | 124.8 |
| Fructooligosaccharides Powder | 120.0 |
| White Sugar | 80.0 |
| Whole Milk Powder | 75.0 |
| Milk Protein Concentrate | 45.9 |
| Calcium Carbonate | 27.9 |
| Magnesium Phosphate | 15.9 |
| Flavor | 5.0 |
| Vitamin/Mineral Premix | 3.5 |
| Sodium Ascorbate | 1.8 |
| Curcumin | 1.0 |
| Vitamin K2 (1% by weight) | 0.07 |

### Examples 4-5

Examples 4 and 5 illustrate exemplary compositions formulated as nutritional powders, the ingredients of which are listed in Table 4 below. All ingredient amounts are listed as kilogram per 1000 kilogram batch of product, unless otherwise specified. A 40 gram serving of the nutritional powder of Example 4 will provide 8.6 grams of protein, 400 milligrams of Curcumin, 100 micrograms of Vitamin K2, 424 milligrams of calcium, and 5 micrograms of Vitamin D3. A 30 gram serving of the nutritional powder of Example 5 will provide 6.45 grams of protein, 300 milligrams of Curcumin, 75 micrograms of Vitamin K2, 318 milligrams of calcium, and 4.8 micrograms of Vitamin D3.

TABLE 4

| Ingredient | Example 2 | Example 3 |
|---|---|---|
| Skim Milk Powder | 498.0 | 498.0 |
| Corn Syrup Solids | 122.2 | 122.2 |
| Fructo-oligosaccharide (FOS) | 118.0 | 118.0 |
| Extra Fine White Sugar | 80.0 | 80.0 |
| Lecithinated Whole Milk Powder | 75.0 | 75.0 |
| Milk Protein Concentrate | 45.0 | 45.0 |
| Calcium Carbonate | 26.5 | 26.5 |
| Magnesium phosphate dibasic | 15.0 | 15.0 |
| Flavor | 5.0 | 5.0 |
| Curcumin | 10.0 | 10.0 |
| Vitamin K2 | 0.0025 | 0.0025 |
| Vitamin D3 (in grams) | 0.125 | 0.160 |
| Vitamin/Mineral Premix¹ | 3.497375 | 3.49734 |
| Sodium Ascorbate | 1.8 | 1.8 |

¹The Vitamin/Mineral Premix includes: ferrous sulfate; zinc sulfate; copper sulfate; manganese sulfate; Vitamin A palmitate; Vitamin E acetate; pyridoxine hydrochloride; folic acid; Vitamin K1; Vitamin B12; and maltodextrin.

### Examples 6-10

Examples 6-10 illustrate exemplary compositions formulated as enteral nutritional compositions, the ingredients of which are listed in Table 5 below. The pH of the enteral nutritional compositions is about 6.5. All ingredient amounts are listed as kg per approximately 1000 kg batch of product, unless otherwise specified.

TABLE 5

| | Example 6 | Example 7 | Example 8 | Example 9 | Example 10 |
|---|---|---|---|---|---|
| Ingredient | kg/1000 kg | kg/1000 kg | kg/1000 kg | kg/1000 kg | kg/1000 kg |
| Water | Q.S. | Q.S. | Q.S. | Q.S. | Q.S. |
| Milk Protein Concentrate | 66.6 | 66.6 | 66.6 | 66.6 | 66.6 |
| Sucrose | 51.5 | 51.5 | 51.5 | 51.5 | 51.5 |
| Maltodextrin | 36.2 | 36.2 | 36.2 | 36.2 | 36.2 |
| Soy Protein Isolate | 15.9 | 15.9 | 15.9 | 15.9 | 15.9 |
| Soy Oil | 13.5 | 13.5 | 13.5 | 13.5 | 13.5 |
| Corn Oil | 5.89 | 5.89 | 5.89 | 5.89 | 5.89 |
| Curcumin | 1.0 | 0.95 | 0.90 | 1.05 | 1.10 |
| Menaquinone-7 (1% by weight) | 0.05 | 0.06 | 0.065 | 0.07 | 0.075 |
| Potassium Citrate | 4.48 | 4.48 | 4.48 | 4.48 | 4.48 |
| Canola Oil | 4.17 | 4.17 | 4.17 | 4.17 | 4.17 |
| Micronized-Tricalcium Phosphate | 2.40 | 2.40 | 2.40 | 2.40 | 2.40 |
| Sodium Citrate | 2.02 | 2.02 | 2.02 | 2.02 | 2.02 |
| Magnesium Chloride | 1.88 | 1.88 | 1.88 | 1.88 | 1.88 |
| Magnesium Phosphate Dibasic | 1.55 | 1.55 | 1.55 | 1.55 | 1.55 |
| Flavoring Agent | 1.50 | 1.50 | 1.50 | 1.50 | 1.50 |
| Sodium Chloride | 1.00 | 1.00 | 1.00 | 1.00 | 1.00 |
| Soy Lecithin | 0.865 | 0.865 | 0.865 | 0.865 | 0.865 |
| Choline Chloride | 0.540 | 0.540 | 0.540 | 0.540 | 0.540 |
| Ascorbic Acid | 0.420 | 0.420 | 0.420 | 0.420 | 0.420 |
| UTM/TM Premix | 0.301 | 0.301 | 0.301 | 0.301 | 0.301 |
| Zinc Sulfate, Monohydrate | 0.068450 | 0.068450 | 0.068450 | 0.068450 | 0.068450 |
| Ferrous Sulfate, Dried | 0.053460 | 0.053460 | 0.053460 | 0.053460 | 0.053460 |
| Manganese Sulfate, Monohydrate | 0.016990 | 0.016990 | 0.016990 | 0.016990 | 0.016990 |
| Cupric Sulfate, Pentahydrate | 0.009248 | 0.009248 | 0.009248 | 0.009248 | 0.009248 |
| Chromium Chloride, Hexahydrate | 0.000573 | 0.000573 | 0.000573 | 0.000573 | 0.000573 |
| Sodium Molybdate, Dihydrate | 0.000463 | 0.000463 | 0.000463 | 0.000463 | 0.000463 |
| Sodium Selencate, Anhydrous | 0.000191 | 0.000191 | 0.000191 | 0.000191 | 0.000191 |
| 45% KOH | 0.250 | 0.250 | 0.250 | 0.250 | 0.250 |
| Potassium Hydroxide | 0.1126 | 0.1126 | 0.1126 | 0.1126 | 0.1126 |
| Carrageenan | 0.350 | 0.350 | 0.350 | 0.350 | 0.350 |
| Water Soluble Vitamin Premix | 0.0727 | 0.0727 | 0.0727 | 0.0727 | 0.0727 |
| Niacinamide | 0.02726 | 0.02726 | 0.02726 | 0.02726 | 0.02726 |
| Calcium Pantothenate | 0.01763 | 0.01763 | 0.01763 | 0.01763 | 0.01763 |
| Thiamine Chloride Hydrochloride | 0.004504 | 0.004504 | 0.004504 | 0.004504 | 0.004504 |
| Pyridoxine Hydrochloride | 0.004337 | 0.004337 | 0.004337 | 0.004337 | 0.004337 |
| Riboflavin | 0.003519 | 0.003519 | 0.003519 | 0.003519 | 0.003519 |
| Folic Acid | 0.000611 | 0.000611 | 0.000611 | 0.000611 | 0.000611 |
| Biotin | 0.0005311 | 0.0005311 | 0.0005311 | 0.0005311 | 0.0005311 |
| Cyanoconbalamin | 0.00001203 | 0.00001203 | 0.00001203 | 0.00001203 | 0.00001203 |
| Vitamin DEK Premix | 0.0644 | 0.0644 | 0.0644 | 0.0644 | 0.0644 |
| dl-Alpha-Tocopheryl Acetate | 0.05392 | 0.05392 | 0.05392 | 0.05392 | 0.05392 |
| Phylloquinone | 0.00008012 | 0.00008012 | 0.00008012 | 0.00008012 | 0.00008012 |
| Vitamin D3 | 0.00001308 | 0.00001308 | 0.00001308 | 0.00001308 | 0.00001308 |
| Vitamin A Palmitate | 0.00825 | 0.00825 | 0.00825 | 0.00825 | 0.00825 |
| Potassium Iodide | 0.000206 | 0.000206 | 0.000206 | 0.000206 | 0.000206 |

### Example 11

In this Example, a study was conducted to determine the effects on bone strength and bone mineral density (BMD) of Curcumin and Vitamin K2, alone and in combination, along with a diet enriched with Vitamin D3 and calcium.

Ovariectomized rats served as the animal model and sham operated rats (without removal of ovaries) served as the control. The ovariectomized rat is a well-known animal model for representing post-menopausal estrogen deficient bone loss in adult humans. Seventy female Sprague-Dawley rats, age about 5 months, were allocated to 7 groups of 6 to 13 animals each, as shown in Table 6 below. Melt-extruded Curcumin (9.35% w/w (400 mg/kg body weight (BW)) and Vitamin K2 (0.2% w/w (500 mg/kg BW)) were administered daily by oral gavage beginning one week before surgery and continuing up to 8 weeks after surgery. An aqueous solution of 0.25% carboxymethyl cellulose (CMC) (5 ml/kg BW) was used as the vehicle control. The animals in the intervention groups were fed modified rat chow diet enriched with calcium carbonate (1%) and Vitamin D3 (1000 IU/kg). The rat chow diet contained approximately 22% by weight crude protein to provide a daily amount of approximately 6 grams of crude protein, which is an adequate amount of protein to meet the nutritional requirements of the rats. The diet regime lasted for 9 weeks, starting 1 week before surgery, and for 8 weeks after surgery. In vivo DXA scans of the lumbar vertebra was carried out before surgery, 3 weeks after surgery, and 8 weeks after surgery. The animals were sacrificed 8 weeks after surgery. The right femur and the fourth and fifth vertebrae (L4, L5) were collected for biomechanical testing and BMD measurement by Micro-CT analysis.

TABLE 6

| | | No. of | | | | |
|---|---|---|---|---|---|---|
| | Model | Animals | Test Article | Dose | Necropsy | Diet |
| Group A | Baseline | 6 | N/A | N/A | One week | Normal |
| | | | | | pre-surgery | |
| Group B | Sham | 6 | 0.25% CMC | 5 ml/kg | 8 weeks | Normal |
| | | | | PO, daily | post-surgery | |
| Group C | Ovariec- | 10 | 0.25% CMC | 5 ml/kg | 8 weeks | Normal |
| | tomized | | | PO, daily | post-surgery | |
| Group D | Ovariec- | 10 | 0.25% CMC | 5 ml/kg | 8 weeks | Normal |
| | tomized | | | PO, daily | post-surgery | (plus Ca and |
| | | | | | | Vitamin D3) |
| Group E | Ovariec- | 12 | Melt-Extruded | 400 mg/kg | 8 weeks | Normal |
| | tomized | | Curcumin | body weight, | post-surgery | (plus Ca and |
| | | | | PO, daily | | Vitamin D3) |
| Group F | Ovariec- | 13 | Vitamin K2 | 500 mg/kg | 8 weeks | Normal |
| | tomized | | | body weight, | post-surgery | (plus Ca and |
| | | | | PO, daily | | Vitamin D3) |
| Group G | Ovariec- | 13 | Melt-Extruded | 400 mg/kg + | 8 weeks | Normal |
| | tomized | | Curcumin + | 500 mg/kg | post-surgery | (plus Ca and |
| | | | Vitamin K2 | body weight, | | Vitamin D3) |
| | | | | PO, daily | | |

Biomechanical Testing—

Biomechanical testing was performed using an MTS 858 test system (MTS System, Minneapolis, Minn.) and its associated software: control system software (793 System SW V0.5E P/N 100-199-969) and Test Work software (TestWorks Servo Hydraulic Application V4.08E P/N 100-189-048). Frozen bone samples, preserved at −200° C., were thawed at 40° C. the night before tests were performed. The flesh was cleaned from excised femurs. Three point bending test of the femoral shaft was conducted with a lower span of 15 mm and force applied at 6 mm/min upon the midpoint of the femoral shaft until the complete break. The load and displacement data were analyzed to identify the maximum load and ultimate load to failure of the femoral shaft.

In Vivo DXA Scan of the Lumbar Vertebrae—

In vivo DXA scans were performed on a QDR X-Ray Bone Densitometer (Hologic, Inc., Waltham, Mass.) using the small animal regional high resolution software with the system. Animals were anesthetized to effect and placed on the scanning platform in a supine position. The region of interest for the analysis was the lumbar vertebrae (L2-L5).

Micro-Computed Tomography (Micro-CT) Analysis—

Right distal femur and fifth lumbar vertebra were scanned for 3-D reconstructed bone volume and bone mineral density (BMD) by a micro-CT system (eXplore Locus, GE, London, Ontario, Canada), at 100 kV and at 27 μm voxel resolution with a field of view (FOV) up to 80 mm in diameter. The Locus system used an X-ray detector with a kV range of 35-80, and a mA range of 0-500. The specific voltage and the current data were automatically set after selecting the resolution and were recorded during sample analysis. Visualization and data reconstruction were performed using the standard eXplore MicroView™ software (GE Medical System) and eXplore Reconstruction Utility software (GE Medical System), respectively. Using these software packages, the area from the cancellous bone area of the distal femur and vertebra were scanned and the trabecular bone volume was derived in proportion to the bone tissue area. The region of interest (ROI) was 1 mm from the growth plate and 2 mm down that included only trabecular bone. For BMD analysis with micro-CT, the machine was calibrated with a phantom to the known calcium/phosphate ratio so that bone mineral content (BMC) was the absolute number of mineral content and BMD was mineral content divided by the total area included in the analysis.

Results—

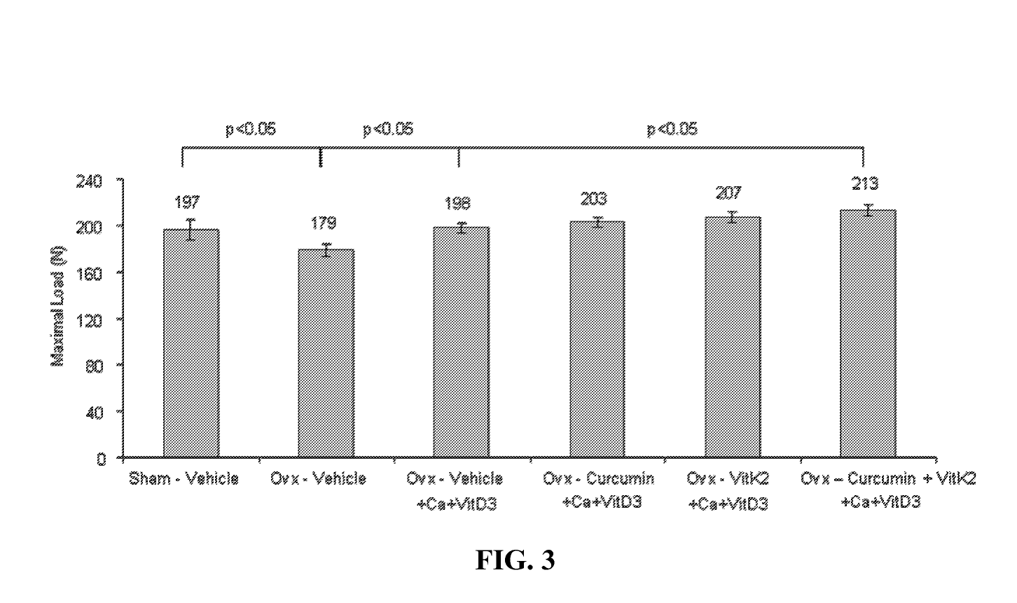

As can be seen from the data, at the time of sacrifice (i.e., 8 weeks after ovariectomization), the rats that received a diet supplemented with Curcumin, Vitamin K2, Vitamin D3, and calcium showed a significant improvement in bone strength and BMD. For example, the Ovx rats that received Curcumin, Vitamin K2, Vitamin D3, and calcium had an average maximal load of the femur of 213 N, which was statistically significant (p<0.05) compared to the maximal load of 198 N measured for the Ovx rats that received Vitamin D3 and calcium, as seen in FIG. 3. Moreover, the difference in maximal load for the Ovx rats that received Vitamin D3 and calcium (198 N) was statistically significant compared to the Ovx rats that received vehicle only.

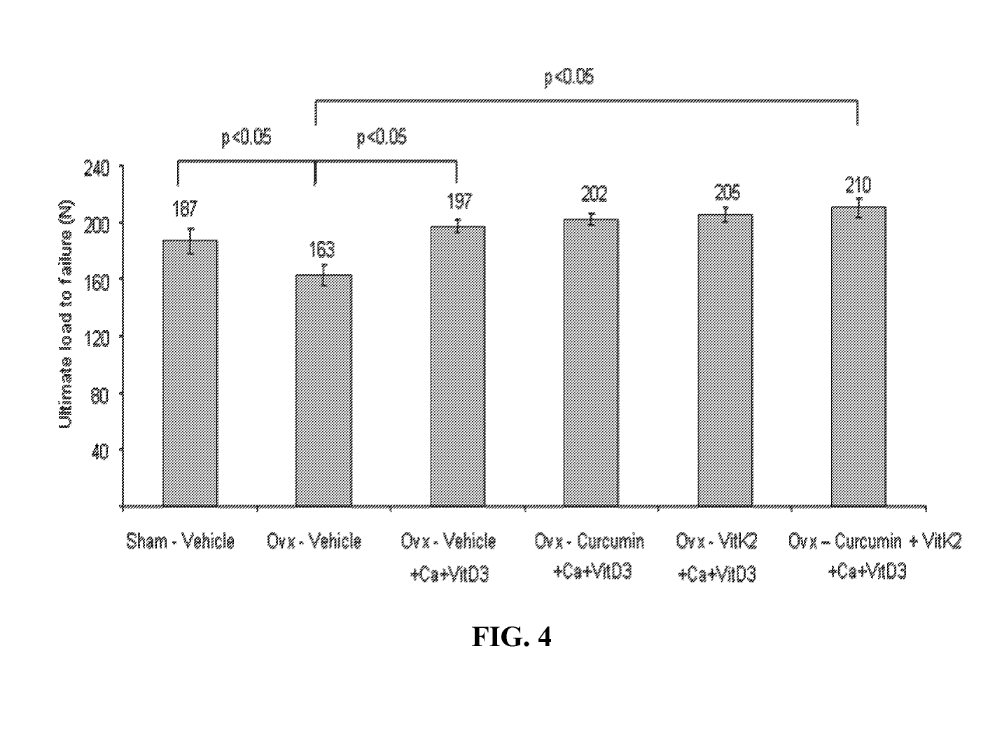

As seen in FIG. 4, the Ovx rats that received Curcumin, Vitamin K2, Vitamin D3, and calcium had an average ultimate load to failure of the femur of 210 N, which was statistically significant (p<0.05) compared to the ultimate load to failure of 163 N for the Ovx rats that received vehicle only. Accordingly, the data of FIGS. 1 and 2 demonstrate that the combination of Curcumin, Vitamin K2, Vitamin D3, and calcium can significantly improve bone strength.

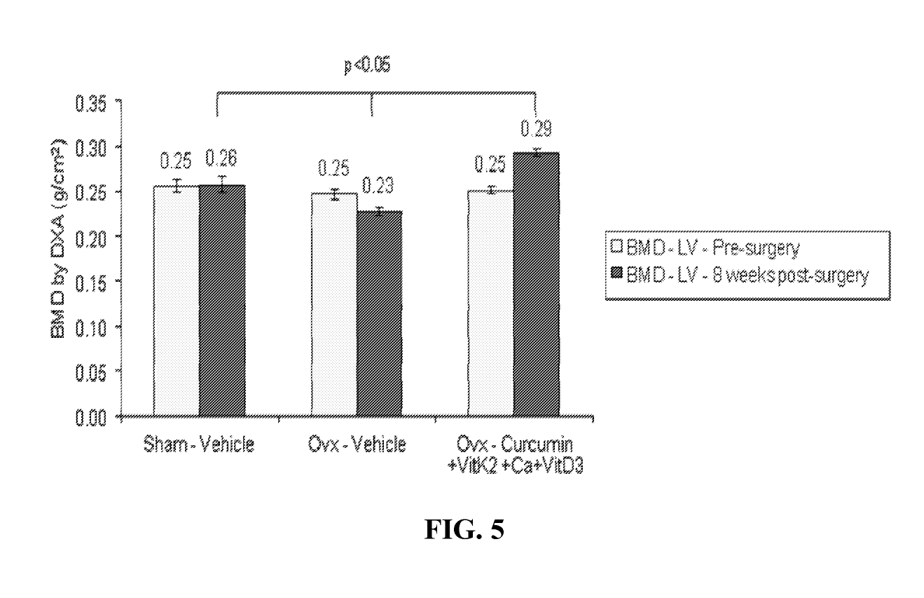

Referring now to FIG. 5, the average BMD of lumbar vertebrae (L4-L5) for each group of rats prior to ovariectomization was 0.25 g/cm². As seen in FIG. 5, the Ovx rats that received Curcumin, Vitamin K2, Vitamin D3, and calcium had an average BMD of lumbar vertebrae of 0.29 g/cm², which was statistically significant (p<0.05) compared to the average BMD of lumbar vertebrae of 0.23 g/cm² for the Ovx rats that received only vehicle.

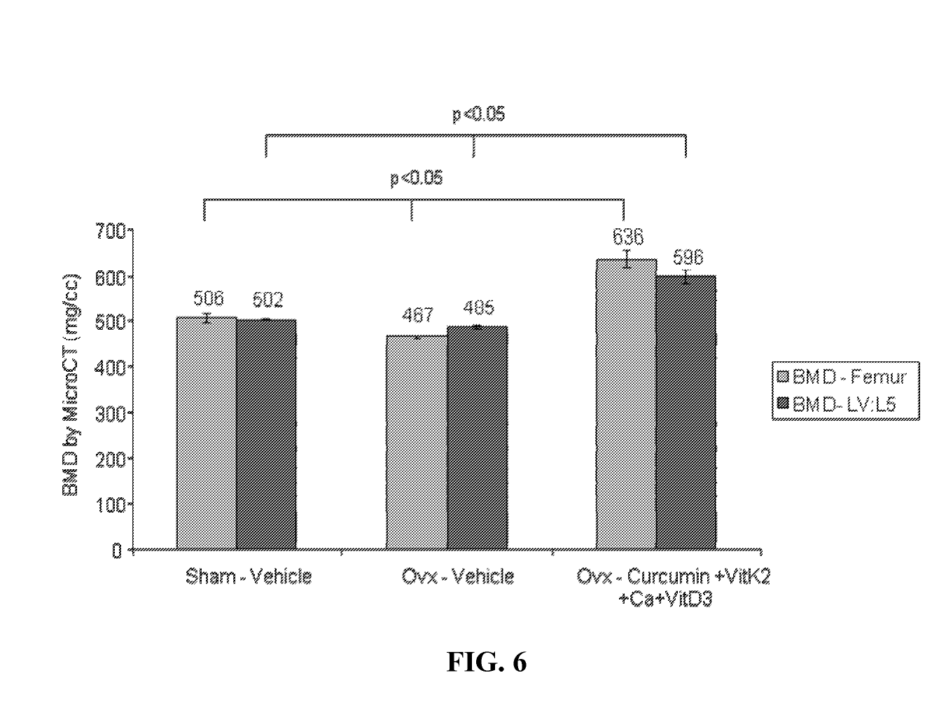

As seen in FIG. 6, the average BMD of the femur and lumbar vertebrae (L5) for Ovx rats that received Curcumin, Vitamin K2, Vitamin D3, and calcium showed a significant improvement with respect to the Ovx rats that received only vehicle. For example, the Ovx rats that received Curcumin, Vitamin K2, Vitamin D3, and calcium had an average femur BMD of 636 mg/cc and an average L5 BMD of 596 mg/cc, which were statistically significant compared to the average femur BMD of 467 mg/cc and average L5 BMD of 485 mg/cc of the Ovx rats that received only vehicle. Accordingly, the data of FIGS. 5 and 6 demonstrate that the combination of Curcumin, Vitamin K2, Vitamin D3, and calcium can significantly improve BMD.

### Example 12

In this Example, a study was conducted to determine the effects on joint health of a combination of Curcumin and Vitamin K2 along with a diet enriched with Vitamin D3 and calcium.

To model postmenopausal osteoarthritis, animals were ovariectomized (Ovx) and joint instability was induced by anterior cruciate ligament tear (ACLT) surgery in the left knee. Thirty-four female Sprague-Dawley rats, age about 5 months, were allocated to 4 groups of 6 to 12 animals each, as shown in Table 7 below. Melt-extruded Curcumin (9.35% w/w (400 mg/kg body weight (BW))) and Vitamin K2 (0.2% w/w (500 mg/kg BW)) were administered daily by oral gavage beginning one week before surgery and continuing up to 8 weeks after surgery. An aqueous solution of 0.25% carboxymethyl cellulose (CMC) (5 ml/kg BW) was used as the vehicle control. The animals in the intervention groups were fed modified rat chow diet enriched with calcium carbonate (1%) and vitamin D3 (1000 IU/kg). The diet regime lasted for 9 weeks, starting 1 week before surgery, and for 8 weeks after surgery. The animals were sacrificed 8 weeks after surgery.

TABLE 7

| | | No. of | | | | |
|---|---|---|---|---|---|---|
| | Model | Animals | Test Article | Dose | Necropsy | Diet |
| Group A | Baseline | 6 | N/A | N/A | One week | Normal |
| | | | | | pre-surgery | |
| Group B | Sham | 6 | 0.25% CMC | 5 ml/kg | 8 weeks | Normal |
| | | | | PO, daily | post-surgery | |
| Group C | Ovariectomized + | 10 | 0.25% CMC | 5 ml/kg | 8 weeks | Normal |
| | ACLT (Ovx + ACLT) | | | PO, daily | post-surgery | (plus Ca and |
| | | | | | | Vitamin D3) |
| Group D | Ovariectomized + | 12 | Melt-Extruded | 400 mg/kg + | 8 weeks | Normal |
| | ACLT (Ovx + ACLT) | | Curcumin + | 500 mg/kg | post-surgery | (plus Ca and |
| | | | Vitamin K2 | body weight, | | Vitamin D3) |
| | | | | PO, daily | | |

Surgery—

Animals were anesthetized with a solution containing Ketamine/Diazepam at a dose of 20-30 mg/kg (i.p.) each. Further anesthesia with 2.0-3.5% isoflurane to effect with a 0.8-1.5 liter flow rate of oxygen was applied during the surgery.

Bilateral ovariectomy was performed from a low abdominal approach. The skin was surgically cleaned, shaved, and incised at the low abdominal midline. The abdominal muscles were incised to enter the abdominal cavity. The freely movable peri-ovarian fat containing the right ovary and uterine horn was grasped with forceps and exteriorized. The uterine horn was occluded with a double knot suture several millimeters caudal to the Fallopian tube. After crushing the ovarian blood vessels with a hemostat, the ligated portion of uterine horn and peri-ovarian fat with the enclosed ovary were cut with a dissecting scissors and removed. The remaining tissue was released from the hemostat and muscle. The incision was closed with single interrupted suture. The entire procedure was repeated on left side. The low abdominal midline skin incision was closed with three or four wound clips. For Sham animals, ovarian tissue was externalized but not excised.

After ovariectomization (Ovx) surgery, the animals were immediately subject to surgically induced joint disability by Anterior Cruciate Ligament Transection (ACLT) in the left knee. The left hind limb of each rat around the knee region was shaved and cleaned with 2% iodine tincture and 75% ethanol. An approximately 1 cm long incision was made to the medial side of the knee on the skin. The patella was displaced outside the groove to expose the anterior cruciate ligament. The ligament was transected by a pair of scissors or surgical blade. The surgical wound was irrigated with sterile saline after the procedures. The fascia was closed with absorbable sutures, while a non-absorbable monofilament suture material was used to close the skin incisions.

Administration—

Vehicle and test articles were administered via oral gavage on a daily basis. The amount of the test articles given was based on the up-to-date body weight, which was measured once per week. The treatment started one week before the surgery, and continued up to 8 weeks after surgery.

Histology Preparation and Histopathology Evaluation—

At the end of 8 weeks post-surgery, all animals were euthanized by CO₂. The left knee of each animal was dissected out and preserved in 10% buffered formalin and stored at room temperature for histology. The left knee joint was decalcified, embedded in paraffin, sectioned, and stained with toluidine blue and Safranin O. The assessment of each joint was carried out using a modified Mankin's scoring system for severity of articular cartilage degeneration and other tissue changes. The modified Mankin's scoring system is shown in Table 8. In the modified Mankin scoring system, a higher score is indicative of a more severely damaged or degenerated joint, while a score that is close to zero is indicative of a normal or non-degenerated joint.

TABLE 8

| Characteristic | Grading | Score |
|---|---|---|
| I. Nature of | Some fibrocartilage, mostly non- | 4 |
| predominant | chondrocytic cells | |
| tissue | Mostly fibrocartilage | 3 |
| | Mixed hyaline and fibrocartilage | 2 |
| | Mostly hyaline cartilage | 1 |
| | Hyaline cartilage | 0 |
| II. Structural | | |
| Characteristics | | |
| A. Surface | Severe disruption, including fibrillation | 4 |
| regularity | Moderate disruption | 3 |
| | Mild fissure | 2 |
| | Superficial horizontal lamination | 1 |
| | Smooth and intact | 0 |
| B. Structural | Severe disintegration | 4 |
| integrity | Moderate to severe disruption | 3 |
| | Moderate disruption, including cysts | 2 |
| | Mild disruption | 1 |
| | Normal | 0 |
| III. Freedom | | |
| from Cellular | | |
| Changes of | | |
| Degeneration in | | |
| Chondrocyte | | |
| A. Chondrocyte | 70%-100% of cells | 4 |
| clustering | 50% to 70% of cells | 3 |
| | 20% to 40% of cells | 2 |
| | <10% of cells | 1 |
| | No clusters | 0 |
| IV. Degenerative | Severe reduced proteoglycan staining and/or | 4 |
| changes in | Chondrocyte degeneration/necrosis | |
| cartilage | Moderate to severe reduced proteoglycan | 3 |
| | staining and/or Chondrocyte | |
| | degeneration/necrosis | |
| | Moderate reduced proteoglycan staining | 2 |
| | and/or Chondrocyte degeneration/necrosis | |
| | Mild reduced proteoglycan staining and/or | 1 |
| | Chondrocyte degeneration/necrosis | |
| | Normal cartilage | 0 |
| V. Joint Area | | |
| A. Inflammatory | Severe | 4 |
| response in | Moderate to severe | 3 |
| joint region | Moderate | 2 |
| | Mild | 1 |
| | None | 0 |
| B. Neo- | Many neovascular channels found in the | 4 |
| vascularization | ligament of the joint | |
| | Some neovascular channels found in the | 3 |
| | ligament of the joint | |
| | Occasional neovascular channels found in | 2 |
| | the ligament of the joint | |
| | One or two neovascular channels found in | 1 |
| | the ligament of the joint | |
| | Normal | 0 |
| VI. Osteophytes | Large axial and abaxial osteophytes | 4 |
| | Large axial or abaxial osteophyte | 3 |
| | Small osteophyte causing shape change to | 2 |
| | plateau and present axially or abaxially | |
| | Small focus of woven bone +/− localized | 1 |
| | blood vessels at axial or abaxial margin | |
| | but no or minimal shape change to plateau | |
| | None | 0 |

| | |
|---|---|
| Maximum Subtotal | 32 |

Results—

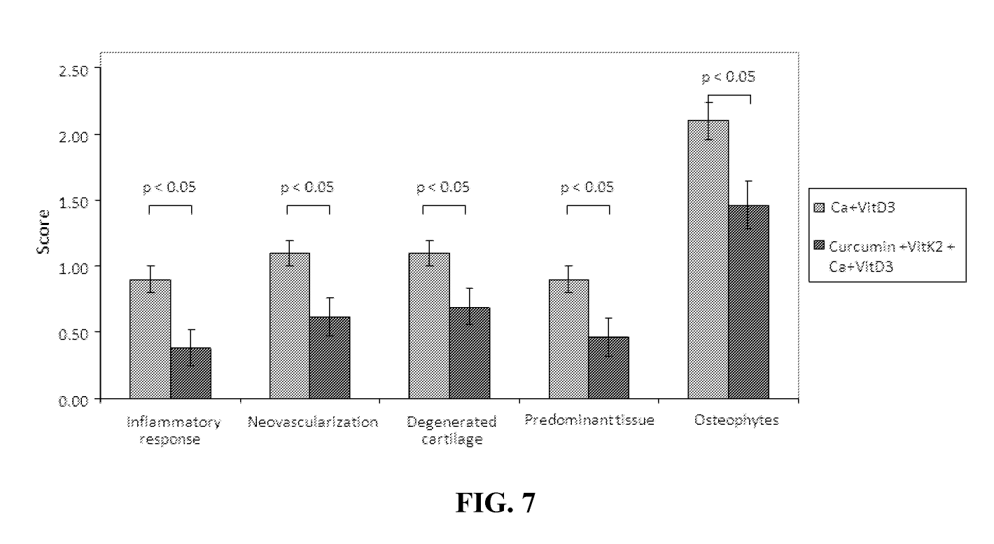

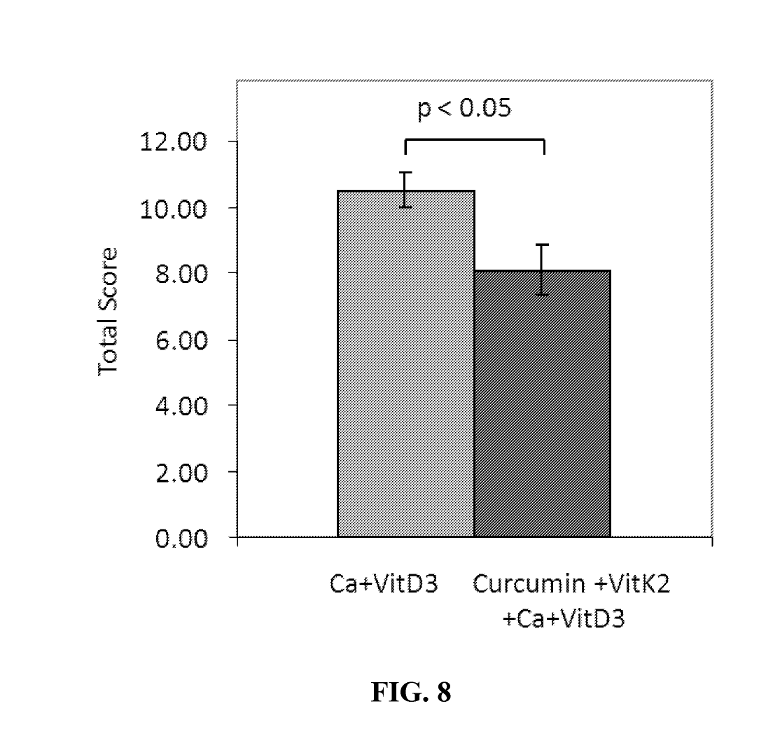

As can be seen from FIGS. 7-9, at the time of sacrifice (i.e., 8 weeks after surgery), the rats that received a diet supplemented with Curcumin and Vitamin K2 showed a significant improvement in joint health. For example, as seen in FIG. 7, the Ovx+ACLT rats that received a combination of Curcumin and Vitamin K2, along with a diet enriched in Vitamin D3 and calcium, had lower Modified Mankin's scores as compared to the Ovx+ACLT rats that received only the CMC vehicle, along with a diet enriched in Vitamin D3 and calcium, in the following categories: inflammatory response (0.9 vs. 0.38); neovascularization (1.1 vs. 0.62); degenerated cartilage (1.1 vs. 0.69); predominant tissue (0.9 vs. 0.46); and osteophytes (2.1 vs. 1.46). Notably, the differences between the Modified Mankin's scores of the Ovx+ACLT rats that received a combination of Curcumin and Vitamin K2, along with a diet enriched in Vitamin D3 and calcium, and the Ovx+ACLT rats that received only the CMC vehicle, along with a diet enriched in Vitamin D3 and calcium, were statistically significant (p<0.05). Moreover, as seen in FIG. 8, the total osteoarthritic score, as represented by the total Modified Mankin's score, was statistically significantly lower (p<0.05) in the Ovx+ACLT rats that received a combination of Curcumin and Vitamin K2 (along with a Vitamin D3 and calcium enriched diet) (total score of 10.5) as compared to the Ovx+ACLT rats that received only the CMC vehicle (along with a Vitamin D3 and calcium enriched diet) (total score of 8.08).

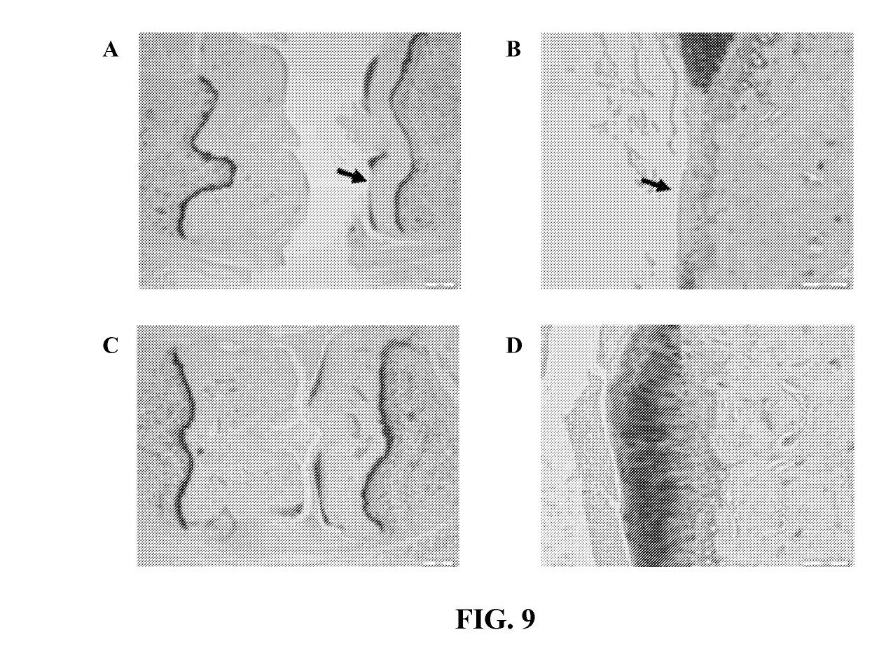

Referring now to FIG. 9, histology images of two knee joints are shown. The histology images of FIGS. 9A and 9B are representative of the Ovx+ACLT rats that received CMC vehicle (along with a Vitamin D3 and calcium enriched diet). FIG. 9B is a magnified image of FIG. 9A, which shows cartilage erosion at the joint (shown with arrow) and other degradative changes of the Ovx+ACLT rat that received the CMC vehicle. On the other hand, the histology images of FIGS. 9C and 9D are representative of the Ovx+ACLT rats that received a combination of Curcumin and Vitamin K2 (along with a Vitamin D3 and calcium enriched diet). FIG. 9D is a magnified image of FIG. 9C, and indicates that Ovx+ACLT rats that received a combination of Curcumin and Vitamin K2 (along with a Vitamin D3 and calcium enriched diet) had less cartilage erosion and other degradative changes as compared to the Ovx+ACLT rats that received the CMC vehicle shown in FIGS. 9A and 9B.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present disclosure illustrates the general inventive concepts by describing various exemplary embodiments thereof, and while such embodiments may be described in considerable detail, the exemplary embodiments are not intended to restrict or in any way limit the scope of the general inventive concepts, including the appended claims, to such detail. Additional advantages and modifications will be readily apparent to those skilled in the art. Therefore, the general inventive concepts, in their broader aspects, are not limited to the specific details, the representative compositions and methods, or the illustrative examples shown and described. Accordingly, departures may be made from such details without departing from the spirit or scope of the general inventive concepts.

1-26. (canceled)

27. A method of improving bone health, joint health, or both in an individual in need thereof, the method comprising:
administering a composition comprising an effective amount of curcumin and an effective amount of vitamin K2 to the individual in need thereof;
whereby the curcumin and the vitamin K2 inhibit osteoclast differentiation to thereby improve bone health, joint health, or both in the individual in need thereof.

28. The method according to claim 27, wherein the composition comprises about 0.001% to about 3.4% curcumin by weight of the composition and about 0.0001% to about 0.1% vitamin K2 by weight of the composition.

29. The method according to claim 27, wherein the curcumin is bioavailable curcumin and the vitamin K2 is menaquinone-7.

30. The method according to claim 27, wherein the composition further comprises vitamin D3 and calcium.

31. The method according to claim 30, wherein the composition comprises, per serving or dose, about 160 IU to about 1,000 IU of vitamin D3 and about 150 milligrams to about 800 milligrams of calcium.

32. The method according to claim 27, wherein the individual in need thereof is a human.

33. The method according to claim 32, wherein the individual in need thereof is a menopausal or postmenopausal woman.

34. The method according to claim 27, wherein the composition further comprises about 1% to about 30% of at least one source of protein by weight of the composition, about 10% to about 80% of at least one source of carbohydrate by weight of the composition, and about 0.5% to about 30% fat by weight of the composition.

35. The method according to claim 34, wherein the composition is a liquid nutritional product.

36. The method according to claim 34, wherein the composition is a reconstitutable powder.

37. A nutritional composition comprising:
about 1% to about 30% of at least one source of protein by weight of the nutritional composition;
about 0.001% to about 3.4% of curcumin by weight of the nutritional composition; and
about 0.0001% to about 0.1% of vitamin K2 by weight of the nutritional composition.

38. The nutritional composition according to claim 37, wherein the nutritional composition further comprises, per serving or dose, about 160 IU to about 1,000 IU of Vitamin D3 and about 150 milligrams to about 1,000 milligrams of calcium.

39. The nutritional composition according to claim 37, wherein the curcumin is bioavailable curcumin and the vitamin K2 is menaquinone-7.

40. A method for maintaining bone quality in an individual in need thereof, the method comprising:
administering to the individual in need thereof a nutritional composition comprising at least one source of protein in an amount sufficient to provide about 5 grams to about 50 grams of protein per serving, and effective amounts of Curcumin, Vitamin K2, Vitamin D3, and calcium;
whereby the bone quality of the subject is maintained upon consumption of the nutritional composition.

41. The method according to claim 40, wherein the nutritional composition is a liquid nutritional product, a reconstitutable powder or a solid nutritional product.

42. The method according to claim 40, wherein the nutritional composition comprises about 0.001% to about 3.4% curcumin by weight of the nutritional composition and about 0.0001% to about 0.1% of vitamin K2 by weight of the composition.

43. The method according to claim 42, wherein the nutritional composition comprises, per serving or dose, about 160 IU to about 1,000 IU of Vitamin D3 and about 150 milligrams to about 800 milligrams of calcium.

44. The method according to claim 40, wherein the individual in need thereof is a human.

45. The method according to claim 44, wherein the individual in need thereof is a menopausal or postmenopausal woman.

46. The method according to claim 44, wherein the individual in need thereof is a human having one or more of osteoarthritis, rheumatoid arthritis, systemic lupus erythematosus, osteopenia, or osteoporosis.
